SEQUENCE LISTING (18 sequences)

SEQ ID NO: 1 - DNA, 1952 bp, Artificial sequence
  pCIn.hG
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgtttaaacttaagcttggtaccgagctcggatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaccctggctccagagagggctagagcggccgctcgagtctagagggcccgtttaaacccgctgatcagcctcgactgtgccttctagttgccagccatctgttgtttgcccctcccccgtgccttccttgaccctggaaggtgccactcccactgtcctttcct

SEQ ID NO: 2 - protein, 211 aa, Artificial sequence
  Synthetic Construct
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 3 - DNA, 836 bp, Homo sapiens
  CDS (201)..(833)
  mat_peptide (432)..(833)
ccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 4 - protein, 211 aa, Homo sapiens; identical to SEQ ID NO: 2

SEQ ID NO: 5 - DNA, 3509 bp, Mus musculus
  CDS (131)..(763)
  mat_peptide (362)..(763)
cggggccccgcgctcctgcccgaggtccggatgggtctcctggatgggattcgggccacttggagttaatgtccaactgggggtctacggagaccggatccgaggtgccgccgccggacgggactctaagatgaagttatgggatgtcgtggctgtctgcctggtgttgctccacaccgcgtctgccttcccgctgcccgccggtaagaggcttctcgaagcgcccgctgaagaccactccctcggccaccgccgcgtgcccttcgcgctgaccagtgactccaatatgcctgaagattatcctgaccagtttgatgacgtcatggattttattcaagccaccattaaaagactgaaaaggtcaccagataaacaagcggcagcgcttcctcgaagagagaggaatcggcaggctgcagctgccagcccagagaattccagagggaaaggtcgcagaggccagaggggcaaaaatcgggggtgcgttttaactgccatacacttaaatgtcactgacttgggtttgggctatgaaaccaaggaggaactgatctttcgatattgcagcggttcctgtgaatcggccgagacaatgtatgacaaaatactaaaaaacctgtctcggagtagaaggctaacaagtgacaaagtaggccaggcatgttgcaggccggtcgccttcgacgacgacctgtcgtttttagatgacaacctggtttaccatattctaagaaagcattccgctaaacggtgtggatgtatctgaccccggctccagagactgctgtgtattgcattcctgctacagtgcgaagaaagggaccaaggttcccaggaaatgtttgcccagagtggaagataaggaccaagatggcggaggcagaggcagaagaagagaaaaagaaggaggaaggtagccaccgtgggagcctggtggaagaaggcccagctacagaaaactggataggagtgagaacagccgtctgagcacttcagggtggcagctgatgtcaccagaagctcagggctggtgtccttacttagctgttggcagtgtttatctgatacagtccatggtcaccgatggcggtccgagtcgtggatgcatgcaaagaaccaagccagtgtatctcctgtggtttgttttcacgtgtttgaagacgccagggaaatgtgacgattccggtgtcacctcttgtcactacattttactgctgaaaataagaaagatgtattttcctgtcactcaatggattcataccctggaaaggaaagggggtggggaggacaaaggcagagacgataaaaagatgacaacctttgaatttgaaaaccggggcctgaggtctattacatccagcattttgggggacgcttggtggttgattctggacacagagtgtgggggtatggagaagttggctaggaacccaaaaaaggctgtcctcatacttagaaacaaacctggaggcatctccttcgtgccctcagaaacaggaagcaagttgtggttttcggcagcattctcataggagagccaatggggaaagcccccagctgccccggccagggagaccccggggcctgttggtttacaaagacggatgttacatacacagctccgttcatgcgtggtcaccagtgaccacagaagctcctcgatgcaatgcatctgtttcaaatacagagatatagagaagatatttattgagattttaagttattattatttattaccattcactaatgaatatcttttttcttcccttatttattaaagtctcttttcaaaggtgccaaagtatatgtgctcacaaaatacaaaggtgacagaggaaatattaacattgagaacgagggtccattctcttcagagtataagagggtttttgctagccaaaaaatcacttactttataaggcagtccttcactaactgccacagatttcagcatttccctccccctctttctgtggacaggatctcctgtagcctaggctgaccttgaacttactgcttggtcaaggacgaccttggccttctgatcctctgacttcctgtggtcttggcatggcagacatcaatcacaccgtatccacaggactaagtatagaattcagggcttcatccatgccaggcaactgtaggtaccaactgggttacacccagcccctgctttctatctgctaaaggaaagggtcaggagggccatctcagctccaccccctcactgggagtaccagtgggactgccacctgtttgcatctcctcttctatccatctgagcagcaaggccccagctccactgcccattgagttcaactctttttcccccttcatcatgggcaatatcataaccacgcattcagaaggctgaactgcccttggaagccctgaacatattgtcacctgaaagtgcaatgaaggcccctgacaggcagccagggtttaacagccccctcaccccccgggggaggtggtacctcttcctctatccttgcccacctgcttcgtccctatggttacacagacaggagaacataggggaactgtgcagggagaggccatctgccttgtcctttgccagggctcagttgtcacctgggctcaacttttgctaccccctcaaacccaatggattccaaggagaaagatggacaggaagagacatctgactggccatagaacaaagaagaggccctccctcattgtttggagggaagaagcagagggtgggaatggcaactctccctgcaccccataatgtccttgtctggcagccaacaaacaggtcatgccttgagtcctatgttagagccttgagtcctatgttacagatttcataaatggaatttttatgtagaagttaatgtatcactccctttgtggctgctatcccccgcccccagagtaggacagatagatctaaaataataaatgaccaattaattggcctctctcgaatagtcatgtcaaattttcaaagtaaccaagaggtaaaagttactaggtatcctttccccttccgtggccctaaagacccacgtttcgcatggttccacaccccctgcaggactgggaagtggagctacgtatgcgctgctctctccgctcctcctgctcaaaattgtgacaacctcagctgcccagcacatttcagatgtcgttccagaccctctgttcactccagagaatcctgaaagatgtggtagctgatgctcccaggtccagacacagcctctagctcttgggggaatccctggagaactcagcatctcgagcaggttcgaatgggagcaggctgccccacccagtccgcagcagggtaaagtttgcgacggttctttactgttgtgatcagcatctgccccagtaaagagcacacttctttggaagttctgacctctctgatgtctcggtcgtttgtgttatacaaccaaagttctctacaaactttatttttgtacaatatcattttgtaactttttacaaataaaagctcatttcta

SEQ ID NO: 6 - protein, 211 aa, Mus musculus
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 7 - DNA, 700 bp, Rattus norvegicus
  CDS (50)..(682)
  mat_peptide (281)..(682)
ggtctacggagaccggatccgaggtgccgccgccggacgggactctaagatgaagttatgggatgtcgtggctgtctgcctggtgttgctccacaccgcgtctgccttcccgctgcccgccggtaagaggcttctcgaagcgcccgccgaagaccactccctcggccaccgccgcgtgcccttcgcgctgaccagtgactccaatatgcccgaagattatcctgaccagtttgatgacgtcatggattttattcaagccaccatcaaaagactgaaaaggtcaccagataaacaagcggcggcacttcctcgaagagagaggaaccggcaagctgcagctgccagcccagagaattccagagggaaaggtcgcagaggccagaggggcaaaaatcgggggtgcgtcttaactgcaatacacttaaatgtcactgacttgggtttgggctacgaaaccaaggaggaactgatctttcgatattgtagcggttcctgtgaagcggccgagacaatgtacgacaaaatactaaaaaatctgtctcgaagtagaaggctaacaagtgacaaggtaggccaggcatgttgcaggccggtcgccttcgacgacgacctgtcgtttttagacgacagcctggtttaccatatcctaagaaagcattccgctaaacggtgtggatgtatctgaccctggctccagaga

SEQ ID NO: 8 - protein, 211 aa, Rattus norvegicus
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 9 - protein, 134 aa, Homo sapiens
SPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 10 - protein, 134 aa, Mus musculus
SPDKQAAALPRRERNRQAAAASPENSRGKGRRGQRGKNRGCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 11 - protein, 134 aa, Rattus norvegicus
SPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 12 - protein, 95 aa, Homo sapiens
GCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 13 - protein, 95 aa, Mus musculus
GCVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 14 - protein, 95 aa, Rattus norvegicus
GCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 15 - DNA, 27 bp, artificial sequence
  PCR primer sequence
ggtctacggagaccggatccgaggtgc

SEQ ID NO: 16 - DNA, 26 bp, artificial sequence
  PCR primer sequence
tctctggagccagggtcagatacatc

SEQ ID NO: 17 - DNA, 1752 bp, artificial sequence
  pNS1n.hGDNF
agatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaccctggctccagagagggctagagcggccgctcgagtctagagggcccgtttaaacccgctgatcagcctcgactgtgccttctagttgccagccatctgttgtttgcccctcccccgtgccttccttgaccctggaaggtgccactcccactgtcctttccta

SEQ ID NO: 18 - protein, 211 aa, artificial sequence; identical to SEQ ID NO: 2
  Synthetic Construct

(19) United States
(12) Patent Application Publication
(10) Pub. No.: US 2008/0286250 A1
(43) Pub. Date: Nov. 20, 2008

(54) Implantable Biocompatible Immunoisolatory Vehicle for Delivery of Gdnf

(76) Inventors: Jens Tornoe, Kobenhavn O (DK); Lars U. Wahlberg, Asnaes (DK)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/091,124

(22) PCT Filed: Oct. 27, 2006
(86) PCT No.: PCT/DK2006/000596

§ 371 Date: May 13, 2008

Related U.S. Application Data
(60) Provisional application No. 60/730,852, filed on Oct. 28, 2005.

(30) Foreign Application Priority Data
Oct. 28, 2005 (DK) PA200501497

(51) Int. Cl. A61K 48/00; A61P 25/16; A61P 27/02

(52) U.S. Cl. 424/93.21

(57) ABSTRACT

The present invention relates to devices comprising a composition of human cells secreting a therapeutically effective amount of GDNF (Glial cell-line-derived neurotrophic factor) encapsulated in a device comprising a core and a semipermeable membrane allowing for the diffusion of GDNF protein. The human cells are from one cell line.

The present application relates to devices containing GDNF secreting cells, which devices may be used for the treatment of Parkinson's Disease, Amyotrophic Lateral Sclerosis, Huntington's Disease and retinopathies. All references cited herein are incorporated by reference.

### PRIOR ART

GDNF is a neurotrophic factor capable of promoting the survival of dopaminergic neurons. In Parkinson's disease, the dopaminergic neurons degenerate. There are ample reports in literature showing that that long-term delivery of GDNF is a feasible way to treat Parkinson's disease. Because GDNF does not readily cross the blood brain barrier, its administration into the central nervous system requires the use of invasive procedures, which may compromise the integrity of the blood brain barrier.

Long-term delivery of GDNF to the central nervous system behind the blood-brain barrier can be accomplished in different ways: continuous infusion using implanted pumps or cannulae, in vivo gene therapy, transplantation of naked cells that have been genetically modified to secrete GDNF, and implantation of a catheter like device comprising cells secreting GDNF encapsulated behind a semi-permeable membrane. The same applies to delivery of GDNF to the eye behind the blood-retina barrier.

Delivery using implanted pumps or cannulae requires repeated infusions into the brain, either through injections via a cannula, or from pumps, which must be refilled every time the reservoir is depleted. Every occasion in which the pump reservoir must be replaced or the injection syringe reinserted through the cannulae represents another opportunity that contaminants might be introduced into the brain, which is especially susceptible to infection. Even with the careful use of sterile procedures, there is risk of infection. It has been reported that even in intensive care units, intracerebroventricular catheters used to monitor intracranial pressure become infected with bacteria after about three days (Saffran, Perspectives in Biology and Medicine, 35, pp. 471-86 (1992)). In addition to the risk of infection, there seems to be some risk associated with the infusion procedure. Infusions into the ventricles have been reported to produce hydrocephaLus (Saffran et al., Brain Research, 492, pp. 245-254 (1989)) and continuous infusions of solutions into the parenchyma is associated with cell necrosis in the brain.

In vivo gene therapy is a promising technique for delivery of protein factors to the central nervous system. It carries the advantage of in-situ synthesis of the acitve GDNF factor. However, gene therapy requires the use of virus vectors which use is inherently associated with risks of insertional mutagenesis and tumorigenesis as well as the inability to stop the GDNF secretion should untoward effects occur.

Transplantation of naked cells, that have been genetically modified to secrete GDNF also has the advantage of local delivery and de novo synthesis of the active factor at the treatment site. However, naked cells integrate into the tissue into which they have been transplanted making a termination of the treatment almost impossible. Furthermore, transplanted naked cells may migrate once inside the brain and establish undesirable populations of GDNF secreting cells in the brain. This is especially true for stem and progenitor cells. As an example of this type of treatment Åkerud et al (J Neurosci 2001, 21:8108-18) describe delivery of GDNF to the mouse striatum using transplantation of naked murine neurosphere cells. Recently, Klein et al (Hum Gen Ther, 2005, 16:509-521) described transplantation of naked human neural progenitor cells transduced with a virus vector coding for GDNF to the lumbar spinal cord of rats with the aim of developing a therapy for ALS (amyotrophic lateral sclerosis).

Encapsulated cell biodelivery combines the advantages of gene therapy while avoiding the drawbacks, as the genome of the patient's cells is not affected, and as the implanted devices can be retrieved if any untoward effect is observed.

Different cell types have been used to deliver GDNF from encapsulated cells to the central nervous system of rodents and primates. E.g. Kishima et al (Neurobiol Dis, 2004, 16:428-39) describes delivery of GDNF to baboon ventricles using encapsulated C2C12 cells. C2C12 cells are murine cells. Tseng et al (J Neurosci, 1997, 17:325-33) describes delivery of GDNF to rat brains using encapsulated BHK cells secreting GDNF. BHK is a hamster cell line.

Although for both BHK and C2C12 cells proof of concept in treatment of PD was provided in rats and baboon disease models, these cells have not been used in clinical studies. The major reason for this is that both BHK and C2C12 cells even if encapsulated are not useful for therapy of human beings, because of the potential risk of triggering an immune-response or zoonosis from the use of xenogeneic cells and the poor long-term viability BHK and C2C12 cells show after encapsulation possibly due to continued proliferation. These cells inherently secrete a number of mouse and hamster proteins respectively, which may be immunogenic to human beings. In any event, BHK and C2C12 cells do not have the required viability when grown under stressed conditions in artificial growth media (U.S. Pat. No. 6,361,771).

Accordingly, there is a need in the art for providing devices with GDNF secreting cells, wherein the GDNF secreting cells are adapted for human therapy, secrete high amounts of GDNF and show long-term stability when implanted into the central nervous system or eye of a mammal, in particular human beings.

### SUMMARY OF THE INVENTION

In a first aspect the invention relates to a biocompatible device comprising

a. an inner core comprising a composition of human cells comprising an expression construct coding for GDNF, and
b. a semipermeable membrane surrounding the composition of cells, said membrane permitting the diffusion of GDNF,
wherein the human cells are from one cell line.

By using human cells the risk of zoonosis and adverse immune reactions is reduced.

By using a cell line it is ensured that all cells in the composition are identical. By having identical cells in the device, the “unit” is more stable as all cells inside the device are identical. Devices are intended to stay in the patient's brain or eye for a very long period, preferably more than 6 months. During such a long period, there may be a certain renewal of cells within the device as some cells inevitably die and undergo necrosis, and may be replaced through spontaneous cell divisions within the device. If the cells in the composition of cells loaded into the device initially represent two or more sub-populations of cells, self-renewal within the device may result in a change in the composition of cells. This in turn may result in a change in GDNF output.

In a particularly preferred embodiment, the cells are capable of phagocytising. In this way debris from dying cells within the device may be removed and the environment within the device may be kept “clean”.

Preferably, the expression construct transfected into the cells is a plasmid vector. For a therapeutic cell-product it is less prefereble to use cells that have been transduced with a virus-vector.

In an even more preferred embodiment of the invention, the expression construct transfected into the cells contains an intron in the transcript. Comparative examples show that the highest levels of GDNF secretion are obtained in cell lines transfected with an expression construct with an intron compared to cell lines with intron-less constructs.

In a further aspect the invention relates to a composition of human cells derived from one cell line, wherein the cells comprise a heterologous expression construct coding for GDNF.

In another aspect the invention relates to a method of treatment of Parkinson's disease comprising implanting into the putamen and/or striatal structures of a subject in need thereof at least one device according to the invention or a composition of cells according to the invention.

In another aspect the invention relates to a method of treatment of Huntington's disease comprising implanting into the putamen and/or striatal structures of a subject in need thereof at least one device according to the invention or a composition of cells according to the invention.

The invention also relates to a method of treatment of Amytrophic Lateral Sclerosis comprising implanting into the intrathecal space and/or the spinal cord of a subject in need thereof at least one device according to the invention or a composition of cells according to the invention. Implantation may be done into the lumbar spinal cord.

Furthermore, the invention relates to a method of treatment of retinopathy, age-related macular degeneration, glaucoma, ocular neovascularisation or retinal degeneration comprising implanting into the eye of a subject in need thereof at least one device according to the invention or a composition of cells according to the invention.

### FIGURES

FIG. 1 shows the GDNF open reading frame inserted into the expression vectors, pNS1n and pCln.hNGF to yield GDNF expression vectors. The translated amino acid sequence is also shown.

FIG. 2 shows the GDNF secretion from selected APRE-19 clones measured by GDNF ELISA. A fixed number of cells were plated. After attachment GDNF secretion was measured in Endothelial Serum-Free Medium after 4 hours incubation. The error bars show standard deviations from mean among different independent measurements. FIG. 2A shows clones transfected with pCln.hG (with intron). FIG. 2B shows clones transfected with pNS1n.hGDNF (without intron).

FIG. 3 shows the GDNF release over 8 weeks of selected ARPE-19 clones growing as confluent cultures. The lower line shows the untransfected parental cell line, ARPE-19. GDNF release was measured by GDNF Elisa in medium after 4 hours incubation. Each line represents one individual clone. FIG. 3A shows clones grown in Human Endothelial Serum-free Medium (Invitrogen). FIG. 3B shows the same clones grown in DMEM/F12 (Invitrogen). Note the different scales of the vertical axis.

FIG. 4A shows the data from week 4 of FIG. 3 for the individual clones. Clones labelled C- are transfected with pCln.hG, which contains an intron. Clones labelled N- are transfected with pNS1n.hGDNF, which does not contain an intron. FIG. 4B shows the average of the two groups of clones for a more direct comparison of the effect of an intron.

FIG. 5 shows a Western Blot of GDNF produced by NGC-0301 (an ARPE-19 clone according to the invention) compared to GDNF purchased from R&D (#212GD; mammalian produced GDNF) and Alomone (#G-240; E. coli produced GDNF). To the left molecular weight markers are indicated. Lanes marked—were not deglycosylated. Lanes marked D were deglycosylated under denaturing conditions. Lanes marked N were deglycosylated under native conditions. For further details, refernce is made to Example 3.

FIG. 6 shows GDNF binding to GFRα1-Ret. The x-axis shows the amount of GDNF (ng/mL). The y-axis shows the extent of ternary complex formation. NGC-0301 represents GDNF from conditioned medium from a cell line according to the invention. R&D represents mammalian recombinant GDNF from R&D systems (#212GD). Alomone represents E. coli recombinant GDNF from Alomone labs (#G-240).

FIG. 7 shows PC12 survival in serum-free medium. The x-axis shows the amount of GDNF (nM). The y-axis shows the Relative MTS reduction +/−SEM. NGC-0301 represents GDNF from conditioned medium from a cell line according to the invention. RED represents mammalian recombinant GDNF from R&D systems (#212GD). Alomone represents E. coli recombinant GDNF from Alomone labs (#G-240).

FIG. 8 shows GDNF release from devices containing GDNF secreting cells measured after incubation for 4 hours in HE-SFM. The results are from devices grown for two weeks in vitro. The devices are PS devices as described in Example 6 (polysulphone membrane, with 90 kDa cutoff, 5 mm long, inner diameter of 700 μm, filled with 50,000 cells). The Y-axis shows the GDNF release as ng GDNF/device/24 hours. The columns represent the average of 5 devices. The bars represent the standard deviation of the average.

FIG. 9 shows GDNF release from devices containing GDNF secreting cells after 8 weeks implantation in rat brains. The GDNF release was measured after 4 hours incubation in HE-SFM and is given as ng GDNF/device/24 hours. The columns are averages of 5 devices. FIG. 6A shows results obtained with PES devices (Polyethersulphone membrane with 280 kDa cutoff, 7 mm long, 500 μm inner diameter, filled with 50,000 cells). FIG. 6B shows results obtained with PS devices (polysulphone membrane with 90 kDa cutoff, 5 mm long, 700 μm inner diameter, filled with 50,000 cells).

FIG. 10 shows a histological section of a representative PES device, that was sectioned after 8 weeks in vivo in a rat brain and subsequent measurement of GDNF output. FIG. 10A shows a section of the whole device with the membrane, the foam support and living cells. FIG. 10B is a close up of a subsection of the device showing excellent cell survival.

### DEFINITIONS

“Introns” refer in this work to those regions of DNA sequence that are transcribed along with the coding sequences (exons) but are then removed in the formation of the mature mRNA. Introns may occur anywhere within a transcribed sequence, between coding sequences of a gene, within the coding sequence of a gene, and within the 5′ untranslated region (5′ UTR) (including the promoter region). Introns in the primary transcript are excised and the exon sequences are simultaneously and precisely ligated to form the mature mRNA. The junctions of introns and exons form the splice sites. The base sequence of an intron conservatively begins with GT and ends with AG in many higher eukaryots.

As used herein “a biocompatible capsule” or “a biocompatible device” means that the device, upon implantation in a host mammal, does not elicit a detrimental host response sufficient to result in the rejection of the device or to render it inoperable, for example through degradation.

As used herein “an immunoisolatory capsule or device” means that the device or capsule upon implantation into a mammalian host minimizes the deleterious effects of the host's immune system on the cells within its core.

Biological activity refers to the biologically useful effects of a molecule on a specific cell. As used herein “a biologically active GDNF” is one which is released or secreted from the cell in which it is made and exerts its effect on a target cell. Biological activity of the secreted GDNF can be verified in the RET-L1 (or RET-L2) Elisa assay and the PC-12 assay described in the examples.

By a “mammalian promoter” is intended a promoter capable of functioning in a mammalian cell.

Down regulation of a promoter means the reduction in the expression of the product of transgene to a level, which may lead to a lack of significant biological activity of the transgene product after in vivo implantation. As used herein “a promoter not subject to down regulation” means a promoter, which, after in vivo implantation in a mammalian host, drives or continues to drive the expression of transgene at a level which is biologically active.

As used herein “long-term, stable expression of a biologically active GDNF” means the continued production of a biologically active GDNF at a level sufficient to maintain its useful biological activity for periods greater than one month, preferably greater than three months and most preferably greater than six months.

A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680)), and the default parameters suggested therein. The ClustalW software is available from as a ClustalW WWW Service at the European Bioinformatics Institute http://www.ebi.ac.uk/clustalw. Using this program with its default settings, the mature (bioactive) part of a query and a reference polypeptide are aligned. The number of fully conserved residues are counted and divided by the length of the reference polypeptide.

The ClustalW algorithm may similarly be used to align nucleotide sequences. Sequence identities may be calculated in a similar way as indicated for amino acid sequences.

### DETAILED DESCRIPTION

### GDNF (Glial Cell-Line Derived Neurotrophic Factor)

GDNF was first described in WO 93/06116. It signals through the RET receptor tyrosine kinase in complex with GFRα1 or GFRα2 co-receptor. The amino acid sequence of the “pre-pro” form of human GDNF is set forth in SEQ ID NO:2, 4, and 17. The amino acid sequence of the “pre-pro” form of mouse GDNF is set forth in SEQ ID NO:6. The amino acid sequence of the “pre-pro” form of rat GDNF is set forth in SEQ ID NO:8.

The nucleotide sequences of the transcripts for human, mouse and rat GDNF can be found in Genbank under accession numbers NM_{—}000514.2, NM_{—}010275.1, and NM_{—}019139.1 respectively. These sequences are also shown in the present application as SEQ ID NO 3, 5, and 7 respectively.

The transcript codes for a pre-pro-precursor. The pre and pro peptides are processed and mature GDNF protein is secreted. The secreted mature GDNF consists of the C-terminal 134 amino acids for both human, mouse and rat GDNF (SEQ ID NO 9, 10, and 11 respectively).

GDNF is a member of the TGF-β superfamily (Massague, et al., 1994, Trends in Cell Biology, 4: 172-178) and a member of glial cell line-derived neurotrophic factor ligand family. The GDNF family includes GDNF, persephin (“PSP”; Milbrandt et al., 1998, Neuron 20: 245253) Neublastin (“NBN”; WO 00/01815) and neurturin (“NTN”; WO 97/08196). The ligands of the GDNF subfamily have in common their ability to induce signalling through the RET receptor tyrosine kinase. The ligands of the GDNF subfamily differ in their relative affinities for a family of neurotrophic receptors, the GFRα receptors. GDNF acts preferably through the GFRα1-RET or GFRα2-RET complexes.

TABLE 1
Amino Acid Sequence Comparison of GDNF (SEQ ID NO 4)
to Persephin, Neurturin, and Neublastin

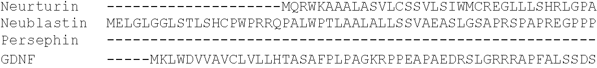

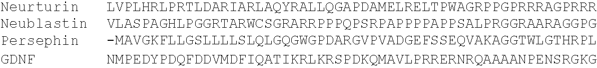

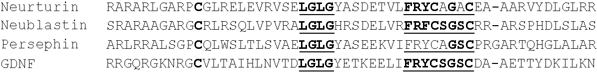

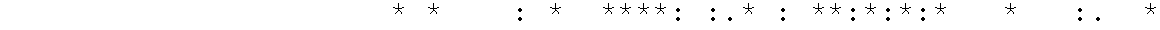

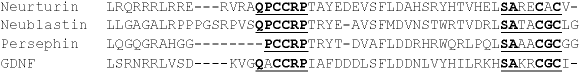

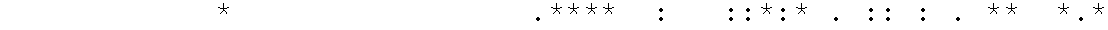

* indicates positions which have a single, fully conserved residue.
: indicates that one of the following ‘strong’ groups is fully conserved: -STA, NEQK, NHQK, NDEQ, QHRK, MILV, MILF, HY, FYW.
. indicates that one of the following ‘weaker’ groups is fully conserved: -CSA, ATV, SAG, STNK, STPA, SGND, SNDEQK, NDEQHK, NEQHRK, VLIM, HFY.

From the amino acid sequence alignment shown in Table 1, it can be seen that GDNF has seven cysteine residues at locations that are conserved within the TGF-[beta] superfamily. Based on this sequence alignment, GDNF contains the GDNF subfamily fingerprint (LGLG-FRYCSGSC-QACCRP-SAKRCGC, the GDNF subfamily fingerprint, underlined in Table 1).

TABLE 2

ClustalW (1.83) multiple sequence alignment of mouse (SEQ ID NO 6), rat
(SEQ ID No 8) and human GDNF (SEQ ID NO 4) orthologues. Conservation is
labelled as in Table 1.

| | |
|---|---|
| GDNF_MOUSE | MKLWDVVAVCLVLLHTASAFPLPAGKRLLEAPAEDHSLGHRRVPFALTSDSNMPEDYPDQ |
| GDNF_RAT | MKLWDVVAVCLVLLHTASAFPLPAGKRLLEAPAEDHSLGHRRVPFALTSDSNMFEDYPDQ |
| GDNF_HUMAN | MKLWDVVAVCLVLLHTASAFPLPAGKRPPEAPAEDRSLGRRRAPFALSSDSNMPEDYPDQ |
| | *************************** ******:***:**.****:************ |
| GDNF_MOUSE | FDDVMDFIQATIKRLKRSPDKQAAALPRRERNRQAAAASPENSRGKGRRGQRGKNRGCVL |
| GDNF_RAT | FDDVMDFIQATIKRLKRSPDKQAAALPRRERNRQAAAASPENSRGKGRRGQRGKNRGCVL |
| GDNF_HUMAN | FDDVMDFIQATIKRLKRSPDKQMAVLPRRERNRQAAAANPENSRGKGRRGQRGKNRGCVL |
| | ********************** *.*************.********************* |
| GDNF_MOUSE | TAIHLNVTDLGLGYETKEELIFRYCSGSCESAETMYDKILKNLSRSRRLTSDKVGQACCR |
| GDNF_RAT | TAIHLNVTDLGLGYETKEELIFRYCSGSCEAAETMYDKILKNLSRSRRLTSDKVGQACCR |
| GDNF_HUMAN | TAIHLNVTDLGLGYETKEELIFRYCSGSCDAAETTYDKILKNLSRNRRLVSDKVGQACCR |
| | *****************************::*** **********.***.********** |
| GDNF_MOUSE | PVAFDDDLSFLDDNLVYHILRKHSAKRCGCI |
| GDNF_RAT | PVAFDDDLSFLDDSLVYHILRKHSAKRCGCI |
| GDNF_HUMAN | PIAFDDDLSFLDDNLVYHILRKHSAKRCGCI |
| | *:***********.***************** |

The GDNF polypeptides secreted by the cell lines of the present invention may be in any bioactive form, including the form of mature proteins, glycosylated proteins, phosphorylated proteins, truncated forms, or any other post-translationally modified protein. It is assumed that a bioactive GDNF is in the dimerized form for each GDNF variant, because dimer formation is required for activity. Little to no activity is observed in a monomeric GDNF polypeptide. A bioactive GDNF polypeptide includes a dimerized polypeptide that, in the presence of a cofactor (such as GFRα1 or GFRα2), binds to RET or to a complex of GFRα1 or 2 and RET, induces dimerization of RET, and autophosphorylation of RET.

The GDNF polypeptides produced by the cell lines of this invention display at least one biological activity of native GDNF. Biological activity for purposes of this invention can be determined by any suitable method. A biologically active GDNF polypeptide is a polypeptide that, when dimerized, can bind, along with GFRα1 or 2, to RET and induce RET dimerization and autophosphorylation (See e.g. Sanicola et al., 1997, Proc. Natl. Acad. Sci. USA, 94:6238). Any method of determining receptor binding and receptor autophosphorylation may be used to evaluate the biological activity the GDNF polypeptide produced by the methods of the invention. For example, the KIRA assay (ELISA) can be used to assess GDNF biological activity. (See also, Sadick et al., 1996, Anal. Biochem., 235(2):207).

GDNF in bioactive form can also be detected using the RetL1 ELISA assay described in example 4. GDNF without biological function will not be detected by the RetL1 ELISA assay.

The following “wild-type” GDNF amino acid (“aa” or “AA”) sequences are exemplary of those secreted GDNF polypeptides that are useful in the methods and compositions of this invention:

AA₁-AA₁₃₄ of SEQ ID NO: 4 or 9 (human mature)
AA₁-AA₁₃₄ of SEQ ID NO: 6 or 10 (mouse mature), and
AA₁-AA₁₃₄ of SEQ ID NO: 8 or 11 (rat mature).

In one embodiment, the preferred GDNF polypeptide contains (seven) cysteines conserved as in SEQ ID NO. 4 at positions 41, 68, 72, 101, 102, 131, and 133. These seven conserved cysteine residues are known within the TGF-superfamily to form three intramonomeric disulfide bonds (contemplated, e.g., in SEQ ID No. 4 between cysteine residues 41-102, 68-131, and 72-133) and one intermonomeric disulfide bond (contemplated, e.g., in SEQ ID NO. 4 between cysteine residues 101-101), which together with the extended beta strand region constitutes the conserved structural motif for the TGF-[beta] superfamily. See, e.g., Daopin et al., Proteins 1993, 17: 176-192.

Preferably the secreted GDNF polypeptide is one of the mature forms of the wild type protein.

The secreted GDNF polypeptides may vary in length. Although the mature human GDNF polypeptide normally consists of the C terminal 134 amino acids of pre pro GDNF, not all of the 134 amino acids are required to achieve useful GDNF biological activity. Amino terminal truncation is permissible (WO 97/11964). Thus, the secreted GDNF polypeptide may contain anywhere from 94 to 134 of the C terminal amino acids of native human GDNF, preferably at least the 95 C-terminal amino acids (corresponding to SEQ ID NO 12). Selection of the exact length of the GDNF polypeptide to be secreted is a design choice, which can be made by one skilled in the art. In addition to varying in length, the secreted human GDNF polypeptide can vary in sequence.

Secreted GDNF polypeptides also include truncated forms of the full-length GDNF molecule. In such truncated molecules, one or more amino acids have been deleted from the N-terminus or the C-terminus, preferably the N-terminus. The truncated GDNF polypeptide may be obtained by using an expression construct coding for a truncated form and an appropriate signal sequence to ensure secretion of the polypeptide.

The truncated GDNF polypeptides described herein preferably include a polypeptide sequence that encompasses the seven cysteine residues conserved in the mature GDNF sequence. In preferred embodiments the truncated GDNF polypeptide includes at least the 95 carboxy terminal amino acids of mature human GDNF (SEQ ID NO 12). Truncated GDNF polypeptides may also contain one or more amino acid substitutions relative to the wild-type sequences as long as the neurotrophic activity is conserved. Thus the invention also includes the use of secreted neurotrophic polypeptides having at least 80% sequence identity to one of the sequences of SEQ ID NO 12; 13, or 14, more preferably at least 85%, more preferably at least 90%, more preferably at least 95%, more preferably at least 98%. Preferably the reference sequence is SEQ ID NO 12.

One truncated form includes the 93 amino acids from the first to the last of the seven cysteine residues of mature GDNF. This corresponds to amino acids no 41 to 133 of SEQ ID No 4.

It is understood that the truncated forms of GDNF disclosed herein (e.g., the 134AA through 95AA forms) have neurotrophic activity.

Truncated forms of the mouse and rat GDNF are also contemplated. These may contain from 94 to 133 of the C-terminal amino acids of SEQ ID No 6 (mouse) or they may contain from 94 to 133 the C-terminal amino acids of SEQ ID No 8 (rat). Preferred truncated mouse and rat sequences are shown in SEQ ID NO 13 and 14.

The GDNF useful in this invention also include those GDNF polypeptides that have an amino acid sequence with substantial similarity or identity to the various prepro, pro, mature and truncated GDNF polypeptides set forth above. Preferably, the secreted GDNF polypeptide has at least 70%, more preferably 85%, still more preferably 90%, or still further preferably 95% identity or similarity to the one of sequences of SEQ ID NO. 9, 10, and 11. Most preferably the secreted GDNF polypeptide has at least 98% similarity or identity to one of the sequences of SEQ ID No. 9, 10, and 11. Preferably to SEQ ID NO 9 (human mature GDNF).

Preferably, any variant GDNF polypeptide does not induce the formation of antibodies against the polypeptide in human beings. Furthermore, it is important that the expression construct encodes a polypeptide that allows for correct processing and folding of the secreted and bioactive GDNF, as misfolding may trigger the formation of antibodies against the protein.

The degree to which a candidate polypeptide shares homology with a GDNF polypeptide of the invention is determined as the degree of similarity or identity between two amino acid sequences.

A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity is determined as described in the definitions section of the present application. Using the ClustalW alignment program, the mature part of a polypeptide may exhibit degree of identity of at Least 70%, more preferably 85%, still more preferably 90%, or still further preferably 95%, most preferably at least 98% with the amino acid sequences presented herein as SEQ ID NO: 4 (human GDNF), SEQ ID NOS: 6 and 8 (rodent GDNF). Preferably the reference sequence is SEQ ID NO 4.

As noted above, the GDNF polypeptides of the invention include variant polypeptides. In the context of this invention, the term “variant polypeptide” includes a polypeptide (or protein) having an amino acid sequence that differs from the mature peptide presented as part of SEQ ID NO. 4 (human GDNF), or SEQ ID No. 6 or 8 (rodent GDNF), at one or more amino acid positions. Such variant polypeptides include the modified polypeptides with conservative substitutions, splice variants, isoforms, homologues from other species, and polymorphisms.

As defined herein, the term “conservative substitutions” denotes the replacement of an amino acid residue by another, biologically similar, residue. Typically, biological similarity, as referred to above, reflects substitutions on the wild type sequence with conserved amino acids. Preferably, a conservative substitution is conservative in the sense defined by the ClustalW alignment program. Positions in the GDNF sequence that may be changed without affecting the bioactivity may e.g. be identified by making a ClustalW alignment of GDNF orthologues (Table 2) and identify non-conserved or semiconserved residues. Positions may also be identified by reference to an alignment of the GDNF family of proteins (Table 1). Residues that are conserved across the GDNF subfamily are likely to be crucial for the bioactivity of the proteins.

In order to avoid the generation of circulating antibodies against GDNF in subjects receiving a encapsulated or naked cells according to the present invention, the GDNF secreted by the cells should be as identical to naturally occurring GDNF in the subject in question. For human beings, this implies that the secreted GDNF preferably consists of the 134 C-terminal amino acids of GDNF (SEQ ID NO 9), glycosylated as GDNF is normally when secreted by human cells.

### Cell Lines

The invention relates to GDNF secreting human cell lines, which have been immortalised by insertion of a heterologous immortalisation gene; to cell lines that are spontaneously immortal; and to growth factor expanded cell lines. In a preferred embodiment of the invention, the human cell line has not been immortalised with the insertion of a heterologous immortalisation gene. As the invention relates to cells which are particularly suited for cell transplantation, preferably as encapsulated cells, such immortalised cell lines are less preferred as there is an inherent risk that they start proliferating in an uncontrolled manner inside the human body and potentially form tumours if they carry known oncogenes.

Growth factor expanded cell lines have the advantage that they depend on added mitogens for continued proliferation. Therefore upon withdrawal of the mitogen prior to or in connection with the filling of a device with cells, the cells stop proliferating and will not proliferate again after implantation into the human body. Some growth factor expanded cell lines may also differentiate upon withdrawal of the mitogen. Growth factor expanded cell lines include stem cells, such as neural stem cells and embryonal stem cells.

Preferably, the cell line is capable of phagocytising. Through phagocytosis the cells will be capable of clearing debris shed by decaying or dying cells within the device.

Preferably, the cell line is a contact inhibited cell line. By a contact inhibited cell line is intended a cell line which when grown in culture flasks as a monolayer under conventional conditions grows to confluency and then substantially stops dividing. This does not exclude the possibility that a limited number of cells escape the monolayer. Inside a capsule or device, the cells grow to confluency and then significantly slow down proliferation rate or completely stop dividing.

A particularly preferred type of cells include epithelial cells which are by their nature contact inhibited and which form stable monolayers in culture.

Even more preferred are retinal pigment epithelial cells (RPE cells). The source of RPE cells is by primary cell isolation from the mammalian retina. RPE cells are capable of phagocytising and are also contact-inhibited cells.

Protocols for harvesting RPE cells are well-defined (Li and Turner, 1988, Exp. Eye Res. 47:911-911; Lopez et al., 1989, Invest. Opthalmol. Vis. Sci. 30:586-588) and considered a routine methodology. In most of the published reports of RPE cell cotransplantation, cells are derived from the rat (Li and Turner, 1988; Lopez et al., 1989). According to the present invention RPE cells are derived from humans. In addition to isolated primary RPE cells, cultured human RPE cell lines may be used in the practice of the invention.

All normal diploid vertebrate cells have a limited capacity to proliferate, a phenomenon that has come to be known as the Hayflick limit or replicative senescence. In human fibroblasts, this limit occurs after 50-80 population doublings, after which the cells remain in a viable but non-dividing senescent state for many months. This contrasts to the behavior of most cancer cells, which have escaped from the controls limiting their proliferative capacity and are effectively immortal.

It is preferable that the cells are capable of undergoing a certain number of cell divisions so they can be genetically modified and expanded to produce enough cells for encapsulated cell therapy or transplantation therapy. Accordingly a preferred cell line is capable of undergoing at least 50 doublings, more preferably at least 60 doublings, more preferably at least 70 doublings, more preferably at least 80 doublings, more preferably at least 90 doublings, such as approximately 100 doublings.

For encapsulation, the cells need to be able to survive and maintain a functional GDNF secretion at the low oxygen tension levels of the CNS. Preferably the cell line of the invention is capable of surviving at an oxygen tension below 5%, more preferably below 2%, more preferably below 1%. 1% oxygen tension corresponds to the oxygen level in the brain.

To be a platform cell line for an encapsulated cell based delivery system, the cell line should have as many of the following characteristics as possible: (1) The cells should be hardy, i.e. viable under stringent conditions (the encapsulated cells should be functional in the vascular and avascular tissue cavities such as in the central nervous system intraparenchymally or within the ventricular or intrathecal fluid spaces or the eye, especially in the intra-ocular environment). (2) The cells should be able to be genetically modified to express GDNF. (3) The cells should have a relatively long life span (the cells should produce sufficient progenies to be banked, characterised, engineered, safety tested and clinical lot manufactured). (4) The cells must be of human origin (which increases compatibility between the encapsulated cells and the host). (5) The cells should exhibit greater than 80% viability for a period of more than one month in vivo in the device (which ensures long-term delivery). (6) The encapsulated cells should deliver an efficacious quantity of GDNF (which ensures effectiveness of the treatment). (7) When encapsulated, the cells should not cause a significant host immune reaction (which ensures the longevity of the graft). (8) The cells should be non-tumourigenic (to provide added safety to the host, in case of device leakage).

In a screening and characterisation of several cell lines it has been found that the ARPE-19 cell line (Dunn et al., 62 Exp. Eye Res. 155-69 (1996), Dunn et al., 39 Invest. Opthalmol. Vis. Sci. 2744-9 (1998), Finnemann et al., 94 Proc. Natl. Acad. Sci. USA 12932-7 (1997), Handa et al., 66 Exp. Eye. 411-9 (1998), Holtkamp et al., 112 Clin. Exp. Immunol. 34-43 (1998), Maidji et al., 70 J. Virol. 8402-10 (1996)) has all of the characteristics of a successful platform cell for an encapsulated cell-based delivery system (U.S. Pat. No. 6,361,771, Tao et al). The ARPE-19 cell line was superior to the other cell lines tested.

The ARPE-19 cell line is available from the American Type Culture Collection (ATCC Number CRL-2302). The ARPE-19 cell line is derived from cultures of normal retinal pigmented epithelial (RPE) cells and expresses the retinal pigmentary epithelial cell-specific markers CRALBP and RPE-65. ARPE-19 cells form stable monolayers, which exhibit morphological and functional polarity.

ARPE-19 cells may be cultured in Complete Growth Medium, the serum-containing medium recommended by the cell depositor. Complete Growth Medium is either a 1:1 mixture of Dulbecco's modified Eagle's medium and Ham's F12 medium with 3 mM L-glutamine, 90%; foetal bovine serum, 10% or a 1:1 mixture of Dulbecco's modified Eagle's medium and Ham's F12 medium with HEPES buffer containing 10% fetal bovine serum, 56 mM final concentration sodium bicarbonate and 2 mM L-glutamine. The cells are preferably incubated at 37° C. in 5% CO₂. The cells are typically plated and grown in Falcon tissue culture treated 6 or 12-well plates or T25 or T75 flasks.

For subculturing, medium is removed, and the ARPE-19 cells are preferably rinsed with 0.05% trypsin, 0.02% EDTA solution, and the trypsin is removed. One to two ml of additional trypsin solution is added. The culture is incubated at room temperature (or at 37° C.) until the ARPE-19 cells detach. A subcultivation ratio of 1:3 to 1:5 is recommended.

The hardiness of candidate cell lines for encapsulated cell therapy can be tested using the following three-step screen. (a) Cell viability screen (The cells may be evaluated under stressed conditions using artificial aqueous humor (aAH) medium or artificial cerebral spinal fluid (aCSF) medium). (b) In vitro ECM screen (The cells may be evaluated in an in vitro extra-cellular matrix (ECM) screen). (c) In vivo device viability screen (The encapsulated cells may be evaluated in an in vivo membrane screen). A detailed description of the screens and results with several human and non human cell lines are found in U.S. Pat. No. 6,361,771 (incorporated by reference).

In the three types of screens described above, ARPE-19 cells has proven superior to a number of other cell lines tested (see U.S. Pat. No. 6,361,771). In particular it should be noted that BHK and C2C12 cells which have been used in the prior art to secrete GDNF did not pass the cell viability screen.

In another embodiment the cell line is selected from the group consisting of: human immortalised fibroblast cell lines, human immortalised mesencymal stem cell Lines, human immortalised astrocyte cell lines, human immortalised mesencephalic cell lines, and human immortalised endothelial cell lines, preferably immortalised with SV40T, vmyc, or the catalytic subunit of telomerase (TERT).

Another type of preferred human cells according to the invention are immortalised human astrocyte cell lines. These cell lines may also have the properties required for the uses according to the present invention. The method for generating an immortalised human astrocyte cell lines has previously been described (Price T N, Burke J F, Mayne L V. A novel human astrocyte cell line (A735) with astrocyte-specific neurotransmitter function. In Vitro Cell Dev Biol. Anim. 1999 May; 35(5):279-88). This protocol may be used to generate astrocyte cell lines.

In order to generate monoclonal cell lines, cells that have been genetically modified to secrete GDNF are seeded under conditions allowing only survival of transfected cells as described in Example 1. After selection of surviving cells or colonies, these may be expanded to form compositions of monoclonal cell lines. Generation of monoclonal cell lines can also be generated using limited dilution, which method requires test of every single selected clone, as there is no selection of transfected cells.

The monoclonal cell lines can subsequently be subjected to selection for high secretion of GDNF, to in vitro and in vivo long term stability screening, before a suitable clone is selected. A selected monoclonal cell line may be further subjected to safety testing and cell banking before it is used for human therapy.

### Long Term Stability

Preferably the cell lines used in the present invention are capable of surviving for extended periods (several months and up to one year or more) when transplanted as encapsulated cells in vivo. The cell lines are preferably also capable of maintaining a secretion of bioactive GDNF at a level sufficient to ensure the therapeutic efficacy for a period greater than one month, preferably greater than three months, more preferably greater than six months. It is also preferable that the cells are capable of maintaining a relevant secretion of bioactive GDNF after encapsulation for at least one month, more preferably at least three months, more preferably at least six months. As shown in Example 8, GDNF secretion and viability remained high after two months implantation in the brain of normal rats.

The level of secretion preferably is at least 0.5 ng biologically active GDNF per 10⁵ cells per 24 hours is at least 0.5 ng, more preferably at least 0.75 ng, more preferably at least 1 ng, more preferably at least 2 ng, more preferably at least 2.5 ng, more preferably at least 5 ng, more preferably at least 7.5 ng, more preferably at least 10 ng, more preferably at least 15 ng, more preferably at least 20 ng, more preferably at least 25 ng, more preferably at least 50 ng. As evidenced by the appended examples, several human contact inhibited clones secreting in excess of 5 and even 10 ng/10⁵ cells/24 hours have been established.

When measured on a device level, the device (comprising encapsulated cells) is preferably capable of secreting in excess of 0.1 ng biologically active GDNF per 24 hours. More preferably, the amount of biologically active GDNF per 24 hours per device is at least 1 ng, more preferably at least 2 ng, more preferably at least 2.5 ng, more preferably at least 5 ng, more preferably at least 7.5 ng, more preferably at least 10 ng, more preferably at least 15 ng, more preferably at least 20 ng, more preferably at least 25 ng. These numbers refer to cylindrical devices of 5-7 mm length having a inner diameter of 500-700 μm and being loaded with 50000 cells.

### Deposit

Two preferred Homo sapiens cell lines have been deposited by NsGene A/S, Baltorpvej 154, DK-2750 Ballerup, Denmark, under the Budapest Treaty with DSMZ, Mascheroder Weg 1b, D-38124 Braunschweig, Germany on Sep. 21, 2005 under accession number: DSM ACC2733 (NGC-0363) and DSM ACC2732 (NCG-0301). Both deposited cell lines are ARPE-19 clones transfected with pCln.hG as defined herein. The deposited cell lines are identified in the examples as C101 (NCG-0301) and C63 (NGC-0363).

### Expression Vectors

Construction of vectors for recombinant expression of GDNF for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982).

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue specific regulatory sequences). It will be appreciated by those skilled in the art that the design of the expression vector can depend on such factors as the choice of the host cell to be transformed, the level of expression of protein desired, etc. The expression vectors used in the invention can be introduced into host cells to thereby produce GDNF and GDNF mutants and variants encoded by nucleic acids as described herein.

In a preferred embodiment of the invention, the cells are transfected with a non-viral expression vector. The use of a non-viral expression vector is preferred for reasons of safety once the cells are implanted into a recipient subject.

In a preferred embodiment, the expression vector is a mammalian plasmid expression vector. Examples of mammalian plasmid expression vectors include pCDM8 (Seed, 1987. Nature 329: 840), pCl (Promega Inc), pSI (Promega), pNS (Example 1), pUbi1z (Johansen et al 2003, J Gene Medicine, 5:1080-1089), and pMT2PC (Kaufman, et al., 1987. EMBO J. 6: 187 195). When used in mammalian cells, the expression vector's control functions are often provided by viral regulatory elements. For other suitable expression systems for eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters and the UbiC promoter derived from human ubiquitin C (WO 98/32869).

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including cottagen type I (1 and 2) (Prockop and Kivirikko, N. Eng. J. Med. 311: 376 (1984); Smith and Nites, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, Mt1 promoter, and Elongation Factor 1 alpha promoter (EF-1alpha). A particularly preferred promoter is one which is not subject to down regulation in vivo.

Examples of inducible/repressible promoters include: Tet-On, Tet-Off, Rapamycin-inducible promoter, Mx1.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1:457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Kozak consensus sequence, Woodchuck hepatitis virus post-transcriptional regulation element, WPRE, SP163 enhancer, CMV enhancer, non-translated 5′ or 3′ regions from the tau, TH or APP genes, and Chicken [beta]-globin insulator or other insulators. Preferable enhancing elements include Kozak consensus sequence, WPRE and beta-globin insulator.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV.

A special and preferred type of retroviruses includes the lentiviruses which can transduce a cell and integrate into its genome without cell division. A lentivirus particle can be produced from a lentiviral vector comprising a 5′ lentiviral LTR, a tRNA binding site, a packaging signal, a promoter operably linked to a polynucleotide signal encoding GDNF, an origin of second strand DNA synthesis and a 3′ lentiviral LTR.

Retroviral vectors are the vectors most commonly used in human clinical trials, since they may carry 7-8 kb of heterologous DNA and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the cell's genome.

Three classes of retroviral particles have been described; ecotropic, which can infect murine cells efficiently, and amphotropic, which can infect cells of many species. The third class includes xenotropic retrovirus, which can infect cells of another species than the species which produced the virus. Their ability to integrate only into the genome of dividing cells has made retroviruses attractive for marking cell lineages in developmental studies and for delivering therapeutic or suicide genes to cancers or tumours.

The retroviral vectors preferably are replication defective. This prevents further generation of infectious retroviral particles in the target tissue—instead the replication defective vector becomes a “captive” transgene stably incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the □ signal, and expressing the gag/pol genes and the env gene from separate transcriptional units. In addition, in some 2. and 3. generation retroviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses. See, e.g., U.S. Pat. No. 4,861,719, herein incorporated by reference.

### Introns

In a preferred embodiment of the invention, the expression construct coding for GDNF or a GDNF variant includes an intron in the transcript. The highest producing cell lines have been obtained with intron-containing expression constructs.

From an analysis of the human genome at GenBank it can be derived that the smallest known intron is 4 bp and the longest known intron is 1,022,077 bp. Based on this knowledge, it is contemplated that the length of the intron used in the context of the present invention can be varied considerably. Except from the upper known limit given by Genbank, it is difficult to give any upper limit for the length of an intron, which is functional in the context of the present invention. In the broadest possible context there is no upper limit for the length of the intron, as long as it can be successfully cloned into the expression vector. For practical reasons one of skill in the art would select an intron, which is less than 100,000 bp long, more preferably less than 10,000 bp long.

The only parts of an intron that are really highly conserved are the sequences immediately within the intron. This identifies the formula of a generic intron as:

### GT . . . AG

The ends are named proceeding from left to right along the intron, that is as the left (or 5′) and right (or 3′) splicing sites. Sometimes they are referred to as the donor and acceptor sites. The bases immediately adjacent the donor and acceptor sites are less conserved. The frequency of different bases at specific positions relative to the splicing sites follow the following percentages (Lewin B, Genes V, Oxford University Press, Oxford, 1994, page 914):

Exon Intron Exon
A G G T A A G T------C A G N
64% 73% 100% 100% 62% 68% 84% 63% 65% 100% 100%

The sequence within these splicing sites can for every single intron be varied considerably by addition, deletion or substitution of bases. In a preferred embodiment of the invention the intron comprises a nucleotide sequence which is derived from a naturally occurring intron, and which has at least 50% sequence identity to said naturally occurring intron. More preferably, the intron has at least 60% sequence identity to said naturally occurring intron, more preferably at least 70%, more preferably at least 75%, more preferably at least 80%, more preferably at least 85%, more preferably at least 90%, more preferably at least 95%, more preferably at least 98%. The higher % sequence identities are preferred as less work is required to assemble to expression construct, and as the possibility of changing the function of the intron increases with the number of differences between a naturally occurring intron and a variant thereof.

In a preferred embodiment the intron is shorter such as less than 10,000 bp, which will considerably ease the cloning. Accordingly the intron may be less than 9,000 bp long, preferably less than 8,000, more preferably less than 7,000, more preferably less than 6,000, more preferably less than 5,000, more preferably less than 4,500, more preferably less than 4,000, more preferably less than 3,500, more preferably less than 3,000, more preferably less than 2,500, more preferably less than 2,000, more preferably less than 1,500, more preferably less than 1,000, such as less than 750, for example less than 500, such as less than 250, for example less than 200.

Similarly, it is expected that the intron should have a certain length to be properly spliced out from the transcript before translation. Therefore preferably the intron is more than 4 bp long, such ad more than 10 bp long, for example more than 20 bp long, preferably more than 50 bp long, more preferably more than 75 bp long.

An intron may be from 4 bp to 1 mio bp long, more preferably from 10-10,000 bp, more preferably from 20-2000 bp, for example from 50-1500 bp, such as preferably from 75-200 bp, for example preferably from 500-1500 bp. The preferred introns of the present invention lie in the range from 100 to 1000 bp.

The origin of the intron may be any. It may also be a synthetic intron as long as it functions as an intron. As the present invention concerns human cell lines, it is preferable to use an intron from a species that is as closely related to human beings as possible. Therefore, preferably the intron is of eukaryotic origin. More preferably the intron is of mammalian origin. For example the intron may be of rodent origin or of primate origin. Still more preferably, the intron is of human origin.

It is preferred to have the intron Located in the 5′ UTR or in the part of the coding sequence closest to the start codon, i.e. the first part of the coding sequence. Cloning is easier when the intron is placed in the 5′ UTR of the transcript. It is contemplated by the present inventors that a similar effect may be obtained by cloning an intron into the sequence coding for GDNF. In the case of cloning inside the coding sequence, the intron is preferably placed in the part of the coding sequence closest to the start codon, i.e. the first part of the coding sequence.

In a preferred embodiment the intron is derived from a first intron. A first intron is the intron located closest to the transcription start site in the gene from which it is derived. While first introns are preferred, it is to be understood that any intron such as a second, third, fourth, fifth, or sixth intron may also be used. A first intron of a particular gene may be referred to as intron A.

It is expected that it is sufficient to include one intron in the expression constructs in the human cell lines of the present invention. Including further introns is of course possible, and is also contemplated by the present inventors. In principle there is no upper limit to the number of introns inserted into the transcript but for practical reasons, the skilled practitioner would choose to keep the number as low as possible to keep the length of the expression construct within practical limits. The number of introns may be two, three, four, five or even higher.

One particularly preferred intron is the chimeric intron included in the pCl expression vector available from Promega Corp, Madison Wis., USA (Catalogue no.: E1731). This intron is composed of the 5′-donor site from the first intron of the human b-globin gene and the branch and 3′-acceptor site from the intron that is between the leader and the body of an immunoglobulin gene heavy chain variable region (Bothwell et al, 1981, Cell 24:625). The sequences of the donor and acceptor sites along with the branchpoint site have been changed to match the consensus sequences for splicing (Senaphthy et al, 1990, Meth. Enzymol. 183:252). The pCl expression vector is available from Promega Corp. The length of the intron is 113 bp and the sequence of the intron is set forth in SEQ ID No 1 (bases no 890-1022). The sequence lying between the splice sites of the “pCl intron” can be varied. Preferably the intron comprises a sequence, which is derived from the “pCl intron”, which derived sequence has at least 50% sequence identity to the sequence set forth above. More preferably, the intron comprises a sequence having at least 60% sequence identity to said “pCl intron”, more preferably at least 70%, more preferably at least 75%, more preferably at least 80%, more preferably at least 85%, more preferably at least 90%, more preferably at least 95%, more preferably at least 98%.

Another preferred intron is derived from insulin. Preferably rodent insulin II, more preferably rat preproinsulin II intron A (bases no. 982 . . . 1100 of GenBank acc. # J00748). The sequence lying between the splice sites of rat insulin II intron A can be varied. Preferably the intron comprises a sequence, which is derived from rat insulin Ii A intron and therefore has at least 50% sequence identity to the sequence set forth above. More preferably, the intron comprises a sequence having at least 60% sequence identity to said sequence, more preferably at least 70%, more preferably at least 75%, more preferably at least 80%, more preferably at least 85%, more preferably at least 90%, more preferably at least 95%, more preferably at least 98%.

A further preferred intron is the ubiquitin promoter intron, preferably the human ubiquitin C promoter intron (Johansen et al. 1990, FEBS Lett. 267, 289-294). The UbiC intron is 811 bp long (bases no. 3959.4769 of GenBank acc # D63791). The ubiquitin C promoter intron is available from the pubilz expression vector described in Johansen et al 2003, J Gene Medicine, 5:1080-1089. The sequence lying between the splice sites of said ubiqutin intron can be varied. Preferably the intron comprises a sequence, which is derived from rat ubiquitin intron, and which has at least 50% sequence identity to the sequence set forth above. More preferably, the intron comprises a sequence which has at least 60% sequence identity to the sequence of said ubiquitin intron, more preferably at least 70%, more preferably at least 75%, more preferably at least 80%, more preferably at least 85%, more preferably at least 90%, more preferably at least 95%, more preferably at least 98%.

Another preferred intron is the EF-1alpha intron A (bases no. 609.1551 of Genbank accession number: J04617 J04616 Human elongation factor EF-1alpha gene, complete cds). This intron is 943 bp tong. The sequence lying between the splice sites of said EF-1alpha intron A can be varied. Preferably the intron comprises a sequence which is derived from the EF-1alpha intron A, and which has at least 50% sequence identity to the sequence set forth above. More preferably, the intron comprises a sequence which has at least 60% sequence identity to said EF-1alpha intron A, more preferably at least 70%, more preferably at least 75%, more preferably at least 80%, more preferably at least 85%, more preferably at least 90%, more preferably at least 95%, more preferably at least 98%.

Preferably the intron is selected from the group consisting of the pCl intron, the ratINS-intrA, and the Ubiquitin intron; and a sequence variant having at least 80% sequence identity to the sequence of any of said introns.

More preferably the intron is selected from the group consisting of the pCl intron and sequence variants thereof having at least 80% sequence identity to the sequence of said intron.

### Encapsulation

Encapsulated cell biodelivery is based on the concept of protecting cells from the recipient host's immune system by surrounding the cells with a semipermeable biocompatible material before implantation within the host. The invention includes a device in which cells are encapsulated in an immunoisolatory device. An “immunoisolatory device” means that the device, upon implantation into a recipient host, minimises the deleterious effects of the host's immune system on the cells in the core of the device. Cells are immunoisolated from the host by enclosing them within implantable polymeric devices formed by a microporous membrane. This approach prevents the cell-to-cell contact between host and implanted tissues, eliminating antigen recognition through direct presentation. The membranes used can also be tailored to control the diffusion of molecules, such as GDNF, based on their molecular weight. Using encapsulation techniques, cells can be transplanted into a host without immune rejection, either with or without use of immunosuppressive drugs. Useful biocompatible polymer devices usually contain a core that contains cells, either suspended in a liquid medium or immobilised within an immobilising matrix, and a surrounding or peripheral region of permselective matrix or membrane (“jacket”) that does not contain isolated cells, that is biocompatible, and that is sufficient to protect cells in the core from detrimental immunological attack. Encapsulation hinders elements of the immune system from entering the device, thereby protecting the encapsulated cells from immune destruction. The semipermeable nature of the device membrane also permits the biologically active molecule of interest to easily diffuse from the device into the surrounding host tissue.

The device can be made from a biocompatible material. A “biocompatible material” is a material that, after implantation in a host, does not elicit a detrimental host response sufficient to result in the rejection of the device or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics. Preferably the device of this invention will be similar to those described by WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050, incorporated by reference. Such devices allow for the passage of metabolites, nutrients and therapeutic substances while minimizing the detrimental effects of the host immune system. Components of the biocompatible material may include a surrounding semipermeable membrane and an internal cell-supporting scaffolding. Preferably, the recombinant cells are seeded onto the scaffolding, which is encapsulated by the permselective membrane. The filamentous cell-supporting scaffold may be made from any biocompatible material selected from the group consisting of acrylic, polyester, polyethylene, polyvinylalcohol, polypropylene polyacetonitrile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which seeded cells may adhere (WO 98/05304, incorporated by reference). Woven mesh tubes have been used as vascular grafts (WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

The jacket preferably has a molecular weight cutoff of less than 1000 kD, more preferably between 50-700 kD, most preferably between 70-300 kD. The molecular weight cutoff should be selected to ensure that the bioactive GDNF can escape from the device while protecting the encapsulated cells from the immune system of the patient.

The thickness of the jacket typically ties in the range of 2 to 200 microns, more preferably from 50 to 150 microns. The jacket should have a thickness to give the device sufficient strength to keep the cells encapsulated and should with this in mind be kept as thin as possible to take up as little space as possible.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane may be formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

The device can be any configuration appropriate for maintaining biological activity and providing access for delivery of the product or function, including for example, cylindrical, rectangular, disk-shaped, patch-shaped, ovoid, stellate, or spherical. Moreover, the device can be coiled or wrapped into a mesh-like or nested structure. If the device is to be retrieved after it is implanted, configurations, which tend to lead to migration of the devices from the site of implantation, such as spherical devices small enough to travel in the recipient host's blood vessels, are not preferred. Certain shapes, such as rectangles, patches, disks, cylinders, and flat sheets offer greater structural integrity and are preferable where retrieval is desired. A particularly preferred shape is cylinder-shaped as such a shape is easily produced from hollow fibers which can be produced industrially.

When macrocapsules are used, preferably at least 10³ cells are encapsulated, such as between 10³ and 10⁸ cells are encapsulated, most preferably 10⁴ to 10⁷ cells are encapsulated in each device. Of course, the number of cells in each device depends on the size of the device. As a rule of thumb, in a device with foam (described below) the present inventors have found that loading between 10,000 and 100,000 cells per μL of device (volume calculated as the inner volume including support), more preferably from 15,000 to 50,000 cells per μL, more preferably from 20,000 to 30,000 cells per μL. The number of cells to be loaded also depends on the size of the cells.

Dosage may be controlled by implanting a fewer or greater number of devices, preferably between 1 and 10 devices per patient.

A macrocapsule in the present context is a device having a volume of at least 0.5 μL, such as from 1 to 10 μL.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the devices may be used, including the use of polymer adhesives or crimping, knotting and heat sealing. In addition, any suitable “dry” sealing method can also be used, as described, e.g., in U.S. Pat. No. 5,653,687, incorporated by reference.

The encapsulated cell devices are implanted according to known techniques. Many implantation sites are contemplated for the devices and methods of this invention. These implantation sites include, but are not limited to, the central nervous system, including the brain, spinal cord (see, U.S. Pat. Nos. 5,106,627, 5,156,844, and 5,554,148, incorporated by reference), and the aqueous and vitreous humors of the eye (see WO 97/34586, incorporated by reference).

### Foam Scaffolds:

The foam scaffold may be formed from any suitable material that forms a biocompatible foam with an open cell or macroporous structure with a network of pores. An open-cell foam is a reticulate structure of interconnected pores. The foam scaffold provides a non-biodegradable, stable scaffold material that allows attachment of adherent cells. Among the polymers that are useful in forming the foam scaffolds for the devices of this invention are thermoplastics and thermoplastic elastomers.

Some examples of materials useful in forming suitable foam scaffolds are listed in Table 3.

TABLE 3

| Thermoplastic | |
|---|---|
| Thermoplastics: | Elastomers: |
| Acrylic | Polyamide |
| Modacrylic | Polyester |
| Polyamide | Polyethylene |
| Polycarbonate | Polypropylene |
| Polyester | Polystyrene |
| Polyethylene | Polyurethane |
| Polypropylene | Polyvinyl Alcohol |
| Polystyrene | Silicone |
| Polysulfone | |
| Polyethersulfone | |
| Polyvinylidene fluoride | |

Thermoplastic foam scaffolds made from polysulfone and polyethersulfone, and thermoplastic elastomer foam scaffolds made from polyurethane and polyvinyl alcohol are preferred.

The foam must have some (but not necessarily all) pores of a size that permits cells to attach to the walls or surfaces within the pores. The pore size, pore density and void volume of the foam scaffold may vary. The pore shape may be circular, elliptical or irregular. Because the pore shape can vary considerably, its dimensions may vary according to the axis being measured. For the purposes of this invention, at least some pores in the foam should have a pore diameter of between 20-500 μm, preferably between 50-150 μm. Preferably the foregoing dimensions represent the mean pore size of the foam. If non-circular, the pore may have variable dimensions, so long as its size is sufficient to permit adherent cells to attach to the walls or surfaces within the pore. In one embodiment, foams are contemplated having some elliptical pores that have a diameter of 20-500 μm along the minor axis and a diameter of up to 1500 μm along the major axis.

In addition to the foregoing cell permissive pores sizes, preferably a least a fraction of the pores in the foam should be less than 10 μm to be cell impermissive but still provide channels for transport of nutrients and biologically active molecules throughout the foam. Pore density of the foam (i.e., the number per volume of pores that can accommodate cells, as described above) can vary between 20-90%, preferably between 50-70%. Similarly, the void volume of the foam may vary between 20-90%, preferably between 30-70%.

The walls or surfaces of the pores may be coated with an extracellular matrix molecule or molecules, or other suitable molecule. This coating can be used to facilitate adherence of the cells to the walls of the pores, to hold cells in a particular phenotype and/or to induce cellular differentiation.

Preferred examples of extracellular matrix molecules (ECM) that can be adhered to the surfaces within the pores of the foams include: collagen, laminin, vitronectin, polyornithine and fibronectin. Other suitable ECM molecules include glycosaminoglycans and proteoglycans; such as chrondroitin sulfate, heparin sulfate, hyaturon, dermatan sulfate, keratin sulfate, heparan sulfate proteoglycan (HSPG) and elastin.

The ECM may be obtained by culturing cells known to deposit ECM, including cells of mesenchymal or astrocyte origin. Schwann cells can be induced to synthesize ECM when treated with ascorbate and cAMP. See, e.g., Baron-Van Evercooren et al., “Schwann Cell Differentiation in vitro: Extracellular Matrix Deposition and Interaction,” Dev. Neurosci., 8, pp. 182-96 (1986).

In addition, adhesion peptide fragments, e.g., RGD containing sequences (ArgGlyAsp), YIGSR-containing sequences (TyrIleGlySerArg), as well as IKVAV containing sequences (IleLysValAlaVal), have been found to be useful in promoting cellular attachment. Some RGD-containing molecules are commercially available—e.g., PepTite-2000™ (Telios).

The foam scaffolds of this invention may also be treated with other materials that enhance cellular distribution within the device. For example, the pores of the foam may be filled with a non-permissive hydrogel that inhibits cell proliferation or migration. Such modification can improve attachment of adherent cells to the foam scaffold. Suitable hydrogels include anionic hydrogels (e.g., alginate or carageenan) that may repel cells due to charge. Alternately, “solid” hydrogels (e.g., agarose or polyethylene oxide) may also be used to inhibit cell proliferation by discouraging binding of extracellular matrix molecules secreted by the cells.

Treatment of the foam scaffold with regions of a non-permissive material allows encapsulation of two or more distinct cell populations within the device without having one population overgrow the other. Thus non-permissive materials may be used within the foam scaffold to segregate separate populations of encapsulated cells. The distinct populations of cells may be the same or different cell types, and may produce the same or different biologically active molecules. In one embodiment, one cell population produces a substance that augments the growth and/or survival of the other cell population. In another embodiment, multiple cell types producing multiple biologically active molecules are encapsulated. This provides the recipient with a mixture or “cocktail” of therapeutic substances.

The devices of this invention may be formed according to any suitable method. In one embodiment, the foam scaffold may be pre-formed and inserted into a pre-fabricated jacket, e.g., a hollow fiber membrane, as a discrete component.

Any suitable thermoplastic or thermoplastic elastomer foam scaffold material may be preformed for insertion into a pre-fabricated jacket. In one embodiment we prefer polyvinyl alcohol (PVA) sponges for use as the foam scaffold. Several PVA sponges are commercially available. For example, PVA foam sponges #D-3, 60 μm pore size are suitable (Rippey Corp, Kanebo). Similarly, PVA sponges are commercially available from Ivalon Inc. (San Diego, Cailf.). PVA sponges are water-insoluble foams formed by the reaction of aerated Poly(vinyl alcohol) solution with formaldehyde vapor as the crosslinker. The hydroxyl groups on the PVA covalently crosslink with the aldehyde groups to form the polymer network. The foams are flexible and elastic when wetted and semi-rigid when dried.

As an alternative, support a mesh or yarn may be used as described in U.S. Pat. No. 6,627,422.

For easy retrival and for fastening the device to the skull, the device may be equipped with a tether anchor. Similarly, for easy retrieval and fastening to the eye, the device may be equipped with a suture eyelet.

Capsules may be filled as using a syringe as described in the examples. Alternatively, automated or semi-automated filling may be used.

### Microcapsules

In addition to the macrocapsules described above, the GDNF secreting cells of the present invention may be encapsulated in microcapsules or microspheres. Microcapsules or microspheres as defined herein are capsules holding less than 10⁴ cells per capsule. Microcapsules may contain substantially less than 10⁴ cells, such as less than 1000 cells per capsule for example less than 100 cells per capsule, such as less than 50 cells per capsule, for example less than 10 cells per capsule, such as less than 5 cells per capsule. Such microcapsules may be structurally relatively simple in that they contain cells dispersed more or less uniformly inside a matrix. Microcapsules may also be coated to provide a more two-layered structure and to ensure that no cells project through the surface of the microcapsules. As the microcapsules typically are small (diameter typically less than 250 μm, such as less than 150 μm, for example less than 100 μm, such as less than 50 μm, for example less than 25 μm) they can be handled like a liquid suspension and be injected at a treatment site.

### Support Matrix for GDNF Producing Cells

The method of the present invention further comprises culturing of the GDNF producing cells in vitro on a support matrix prior to implantation into the mammalian brain. The preadhesion of cells to microcarriers prior to implantation in the brain is designed to enhance the long-term viability of the transplanted cells and provide long term functional benefit. Methods for culturing cells on a support matrix and methods for implanting said cells into the brain are described in U.S. Pat. No. 5,750,103 (incorporated by reference).

To increase the long term viability of the transplanted cells, the cells to be transplanted can be attached in vitro to a support matrix prior to transplantation. Materials of which the support matrix can be comprised include those materials to which cells adhere following in vitro incubation, and on which cells can grow, and which can be implanted into the mammalian body without producing a toxic reaction, or an inflammatory reaction which would destroy the implanted cells or otherwise interfere with their biological or therapeutic activity. Such materials may be synthetic or natural chemical substances, or substances having a biological origin.

The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and codified collagen, natural and modified polysaccharides, including dextrans and celluloses (e.g., nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix. The matrix material on which the cells to be implanted grow, or with which the cells are mixed, may be an indigenous product of the cells. Thus, for example, the matrix material may be extracellular matrix or basement membrane material, which is produced and secreted by cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors.

Alternatively, if the solid matrix to which the implanted cells are attached is constructed of porous material, the growth- or survival promoting factor or factors may be incorporated into the matrix material, from which they would be slowly released after implantation in vivo.

When attached to the support according to the present invention, the cells used for transplantation are generally on the “outer surface” of the support. The support may be solid or porous. However, even in a porous support, the cells are in direct contact with the external milieu without an intervening membrane or other barrier. Thus, according to the present invention, the cells are considered to be on the “outer surface” of the support even though the surface to which they adhere may be in the form of internal folds or convolutions of the porous support material which are not at the exterior of the particle or bead itself.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead.

Bead sizes may range from about 10 μm to 1 mm in diameter, preferably from about 90 μm to about 150 μm. For a description of various microcarrier beads, see, for example, Fisher Biotech Source 87-88, Fisher Scientific Co., 1987, pp. 72-75; Sigma Cell Culture Catalog, Sigma Chemical Co., St, Louis, 1991, pp. 162-163; Ventrex Product Catalog, Ventrex Laboratories, 1989; these references are hereby incorporated by reference. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### Suicide Systems

The devices of the present invention, which encapsulate GDNF-secreting cells, may be retrieved from the patient when required. As a further safety precaution the cells may be equipped with a suicide system, which ensures that the cells may be selectively killed upon administration of a suitable drug to the patient in question.

The suicide system is particularly preferred for naked cell transplantation according to the present invention, as the possibilities for removing naked cells after transplantation are very limited.

One such suicide system is based on thymidine kinases. By having a built-in suicide system in which a thymidine kinase is expressed constitutively or inducibly, the cells can be killed by administering to the individual a therapeutically effective amount of a nucleoside analog, such as AZT. The nucleoside analogue can be administered if the encapsulated cells start to proliferate in an uncontrolled manner. One may also wish to terminate the treatment simply because there is no need for the GDNF-secreting cells anymore, because termination must be immediate and cannot await surgical removal of the encapsulated cells or because further treatment is by some other route.

In the cases where transplanted or encapsulated cells have been conditionally immortalised before transplantation there is a theoretical risk that the oncogene initiates transcription after transplantation and that the transplanted cells consequently become tumorigenic. Whenever cells are immortalised by transduction with an oncogene under the control of an inducible promoter (e.g. the Tet on-off system, the Mx1 promoter or the like), a thymidine kinase (TK) enzyme coding sequence may be inserted into the vector construct under the control of the same promoter (e.g. by using an IRES construct) or the TK coding sequence may be inserted into another vector with an identical promoter. This ensures that whenever the oncogene is transcribed, the TK is also transcribed and the transduced and tumorigenic cells can be selectively killed by administering a prodrug.

There are several examples of thymidine kinase (TK) genes described in the art. One preferred TK is the HSV-thymidine kinase. Other preferred kinases include Drosophila melanogaster thymidine kinase described in Munch-Petersen et al 2000, J. Biol. Chem. 275:6673-6679. Mutants of this particular kinase are even more preferred as they have decreased LD₅₀ with respect to several nucleoside analogues (WO 01/88106). Another group of preferred thymidine kinases include plant kinases described in WO 03/100045.

### Immunostimulatory Cell Surface Proteins

In one embodiment there is provided encapsulated human cells capable of expressing an immunostimulatory cell surface polypeptide in addition to GDNF or an GDNF variant. These immunostimulatory cell surface expressing cells are particularly useful when encapsulated for implantation in a human patient, because cells escaping from a ruptured device are destroyed by the patient's immune system. A host immune response will not be triggered by the recombinant cells expressing an immunostimulatory cell surface polypeptide in an intact device. In case of a device failure, however, the released cells are effectively eliminated by phagocytes without complement activation or the creation of an immune memory.

In a specific embodiment, a chimeric polypeptide containing the human transferrin receptor membrane domain anchors a human IgG1 Fc to the surface of the cell plasma membrane in a “reversed orientation”, thus mimicking the configuration of IgG during opsonisation. The human IgG1 chimeric polypeptide binds the Fc receptor to activate phagocytes, such as macrophages, but avoids the undesirable characteristics of also activating the complement cascade (“complement fixation”). A chronically activated complement system can kill host cells, and accumulating evidence suggests that this mechanism can cause many degenerative diseases, including inflammation and neurodegenerative diseases. Further details of this embodiment of the invention are described in U.S. Pat. No. 6,197,294.

According to this embodiment the cell line further comprises a construct comprising a promoter operatively linked to a polynucleotide sequence encoding a fusion protein comprising an immunostimulatory cell surface protein linked at the amino terminus to a second cell surface polypeptide, wherein the second cell surface polypeptide comprises a transmembrane region, wherein upon expression, the fusion protein is expressed on the cell surface.

Preferably the immunostimulatory cell surface polypeptide activates phagocytes but does not fix complement. In one embodiment the immunostimulatory cell surface polypeptide is a region of IgG, preferably Fc. The second cell surface polypeptide may be a transferrin receptor hinge region.

### Neurological Disorders

GDNF has been suggested as a therapeutic factor for the treatment of Parkinson's disease, Amyotrophic Lateral Sclerosis, Alzheimer's disease (U.S. Pat. No. 5,731,284), NMDA associated disorders, such as Huntington's Disease (U.S. Pat. No. 5,741,778), sensorineural hearing loss (U.S. Pat. No. 5,837,681), ophthalmic diseases (U.S. Pat. No. 6,299,895), pain, spinal cord injury, stroke, trauma, and epilepsy.

According to this invention, capsular delivery of GDNF, synthesised by human cells in vivo, to the brain ventricles, brain parenchyma, or other suitable CNS location, ranging from 1-1500 ng/day is contemplated. The actual dosage of GDNF can be varied by implanting high or low producing clones, more or less cells or fewer or greater number of devices. We contemplate delivery of 0.1-1500, preferably 1 to 1000, more preferably 10-600, most preferably 50-500, ng GDNF/human/day, for ventricular delivery and 0.1-1500, preferably 10-150 ng GDNF/human/day for parenchymal delivery. For comparison recombinant human methionyl GDNF produced in E. coli (r-metHuGDNF) has been administered intraventricularly as monthly dosages of 25 to 4,000 μg (Nutt et al, 2003, Neurology, 60:69-73). r-metHuGDNF has also been administered as continuous infusions into the posterodorsal putamen at daily dosages of 14.4-43.2 μg/putamen/day (Love et al, 2005, Nature Medicine, vv(7):703-704).

Intraocularly, preferably in the vitreous, we contemplate delivery of 50 μg to 500 ng, preferably from 100 μg to 100 ng, and most preferably from 1 ng to 50 ng per eye per patient per day. For periocular delivery, preferably in the sub-Tenon's space or region, slightly higher dosage ranges are contemplated of up to 1 μg per patient per day.

In one embodiment, genetically modified human cells secreting human GDNF (hGDNF) are encapsulated in semipermeable membranes, and implanted intraocularly, intraventricularly or intraparenchymally in a suitable mammalian host, preferably a primate, most preferably a human.

Accordingly, GDNF-expressing cell lines of the invention are believed to be useful in promoting the development, maintenance, or regeneration of neurons in vivo, including central (brain and spinal chord), peripheral (sympathetic, parasympathetic, sensory, and enteric neurons), and motorneurons. GDNF-expressing cell lines of the invention are utilised in methods for the treatment of a variety of neurologic diseases and disorders. In a preferred embodiment, the cell lines of the present invention are administered to a patient to treat neurological disorders. By “neurological disorders” herein is meant disorders of the central and/or peripheral nervous system that are associated with neuron degeneration or damage or loss of neurons. Specific examples of neural disorders include, but are not limited to, Alzheimer's disease, Parkinson's disease, Huntington's disease, stroke, CNS trauma, ALS, peripheral neuropathies, neuropathic pain, and other conditions characterized by necrosis or loss of neurons or their processes, whether central or peripheral, in addition to treating damaged nerves due to trauma, burns, kidney disfunction, injury, and the toxic effects of chemotherapeutics used to treat cancer or AIDS. For example, peripheral neuropathies associated with certain conditions, such as neuropathies associated with diabetes, AIDS, or chemotherapy may be treated using the formulations of the present invention.

In various embodiments of the invention, GDNF-expressing cell lines are administered to patients in whom the nervous system has been damaged by idiopathic processes, trauma, surgery, stroke and ischemia, infection, metabolic disease, nutritional deficiency, malignancy, or toxic agents, to promote the survival or growth of neurons, or in whatever conditions have been found treatable with GDNF. For example, GDNF-expressing cell lines of the invention can be used to promote the survival or growth of motorneurons that are damaged by trauma or surgery. Also, GDNF-expressing cell lines of the invention can be used to treat degenerative motorneuron disorders, such as amyotrophic lateral sclerosis (Lou Gehrig's disease), Bell's palsy, and various conditions involving spinal muscular atrophy, or paralysis. GDNF-expressing cell Lines of the invention can be used to treat human neurodegenerative disorders, such as Alzheimer's disease and other dementias, minimal cognitive impairment (MCI), Parkinson's disease, epilepsy, multiple sclerosis, Huntington's disease, Down's Syndrome, nerve deafness, and Meniere's disease. GDNF-expressing cell lines of the invention are particularly useful for treating Parkinson's Disease.

Further, GDNF-secreting cell lines or devices of the invention implanted either in the peripheral tissues or within the CNS, are preferably used to treat neuropathy, and especially peripheral neuropathy. “Peripheral neuropathy” refers to a disorder affecting the peripheral nervous system, most often manifested as one or a combination of motor, sensory, sensorimotor, or autonomic neural dysfunction. The wide variety of morphologies exhibited by peripheral neuropathies can each be attributed uniquely to an equally wide number of causes. For example, peripheral neuropathies can be genetically acquired, can result from a systemic disease, or can be induced by a toxic agent. Examples include, but are not limited to, diabetic peripheral neuropathy, distal sensorimotor neuropathy, AIDS-associated neuropathy, or autonomic neuropathies such as reduced motility of the gastrointestinal tract or atony of the urinary bladder. Examples of neuropathies associated with systemic disease include post-polio syndrome; examples of hereditary neuropathies include Charcot-Marie-Tooth disease, Refsum's disease, Abetalipoproteinemia, Tangier disease, Krabbe's disease, Metachromatic leukodystrophy, Fabry's disease, and Dejerine-Sottas syndrome; and examples of neuropathies caused by a toxic agent include those caused by treatment with a chemotherapeutic agent such as taxol, vincristine, cisplatin, methotrexate, or 3′-azido-3′-deoxythymidine.

A therapeutically effective dose of GDNF-secreting cells or devices is administered to a patient. By “therapeutically effective dose” herein is meant a dose that produces the effects for which it is administered or that amount which provides therapeutic effect in a particular administration regimen. Dosage of the GDNF released from the cell lines or devices of the present invention is that needed to achieve an effective concentration of GDNF in vivo, for the particular condition treated, though the dosage varies with the type of GDNF variant, the desired duration of the release, the target disease, the subject animal species and other factors, such as patient condition. The exact dose will depend on the disorder to be treated and the implantation site, and will be ascertainable by one skilled in the art using known techniques. Due to the higher potency of in situ synthesised GDNF compared to administered protein formulations and the improved dosage profile obtainable by the continuous and local release from devices, in general, the GDNF-secreting cell lines or devices of the present invention are administered to give about 0.01 ng GDNF/kg body weight to about 100 μg/kg per day, preferably from 0.02 ng/kg to 10 μg/kg, more preferably 0.03 to 500 ng/kg, and most preferably 0.5 ng/kg to 100 ng/kg. In some embodiments doses of 0.03 to 1.0 ng/kg, more preferably 0.1 to 0.3 ng/kg, are given. In addition, as is known in the art, adjustments for age as well as the body weight, general health, sex, diet, time of administration, drug interaction and the severity of the disease may be necessary, and will be ascertainable with routine experimentation by those skilled in the art. Typically, the clinician will administer GDNF-secreting cell lines or devices of the invention until a dosage is reached that ameliorates, repairs, maintains, and/or, optimally, reestablishes neuron function. The dosis may also be a prophylactic dose which prevents or reduces degeneration of neurons. The progress of this therapy is easily monitored by conventional assays.

### Ophthalmic Disorders.

The GDNF releasing devices and cell lines of the present invention may be used to treat ophthalmic disorders such as described in U.S. Pat. No. 6,436,427 (incorporated by reference). GDNF has shown potential for treatment of retinopathies, therefore in a preferred embodiment, the GDNF releasing devices of the invention are used to treat retinopathies.

In general, devices are implanted into the vitreus humor of the eye to obtain administration to the retina. Devices are preferably inserted into the pars planum of the vitreous humor.

Retinopathy, e.g. diabetic retinopathy, is characterized by angiogenesis and retinal degeneration. Retinopathy includes, but is not limited to, diabetic retinopathy, proliferative vitreoretinopathy, and toxic retinopathy. Retinopathies may be treated by implanting devices intraocularly, preferably in the vitreous. We most prefer delivery into the vitreous for this indication. It may also be desirable to co-deliver one or more anti-angiogenic factors intraocularly, preferably intravitreally.

Uveitis involves inflammation and secondary degeneration that may affect retinal cells. This invention contemplates treating retinal degeneration caused by uveitis, preferably by vitreal or anterior chamber implantation of devices.

Retinitis pigmentosa, by comparison, is characterized by primary retinal degeneration. This invention contemplates treating retinitis pigmentosa by intraocular, preferably vitreal, implantation of devices.

Age-related macular degeneration involves both angiogenesis and retinal degeneration. Age-related macular degeneration includes, but is not Limited to, dry age-related macular degeneration, exudative age-related macular degeneration, and myopic degeneration. This invention contemplates treating this disorder by implanting one or more devices intraocularly, preferably to the vitreous, and/or one or more anti-angiogenic factors intraocularly or periocularly.

Glaucoma is characterized by increased ocular pressure and loss of retinal ganglion cells. Treatments for glaucoma contemplated in this invention include delivery of GDNF that protect retinal cells from glaucoma associated damage, through intraocular, preferably intravitreal implantation.

Ocular neovascularization is a condition associated with many ocular diseases and disorders and accounting for a majority of severe visual loss. For example, we contemplate treatment of retinal ischemia-associated ocular neovascularization, a major cause of blindness in diabetes and many other diseases; corneal neovascularization, which predisposes patients to corneal graft failure; and neovascularization associated with diabetic retinopathy, central retinal vein occlusion, and possibly age-related macular degeneration.

In one embodiment of the present invention, cells of the invention are encapsulated and surgically inserted (under retrobulbar anesthesia) into the vitreous of the eye. For vitreal placement, the device may be implanted through the sclera, with a portion of the device or tether protruding through the sclera. Most preferably, the entire body of the device is implanted in the vitreous, with no portion of the device protruding into or through the sclera. Preferably the device is tethered to the sclera (or other suitable ocular structure). The tether may comprise a suture eyelet, or any other suitable anchoring means (see e.g. U.S. Pat. No. 6,436,427). The device can remain in the vitreous as long as necessary to achieve the desired prophylaxis or therapy. Such therapies for example include promotion of neuron or photoreceptor survival or repair, or inhibition and/or reversal of retinal neovascularization, as well as inhibition of uveal, retinal, and optic nerve inflammation.

### EXAMPLES

### Example 1

### Cloning and Expression of GDNF

Cloning of GDNF from caudate nucleus polyA mRNA

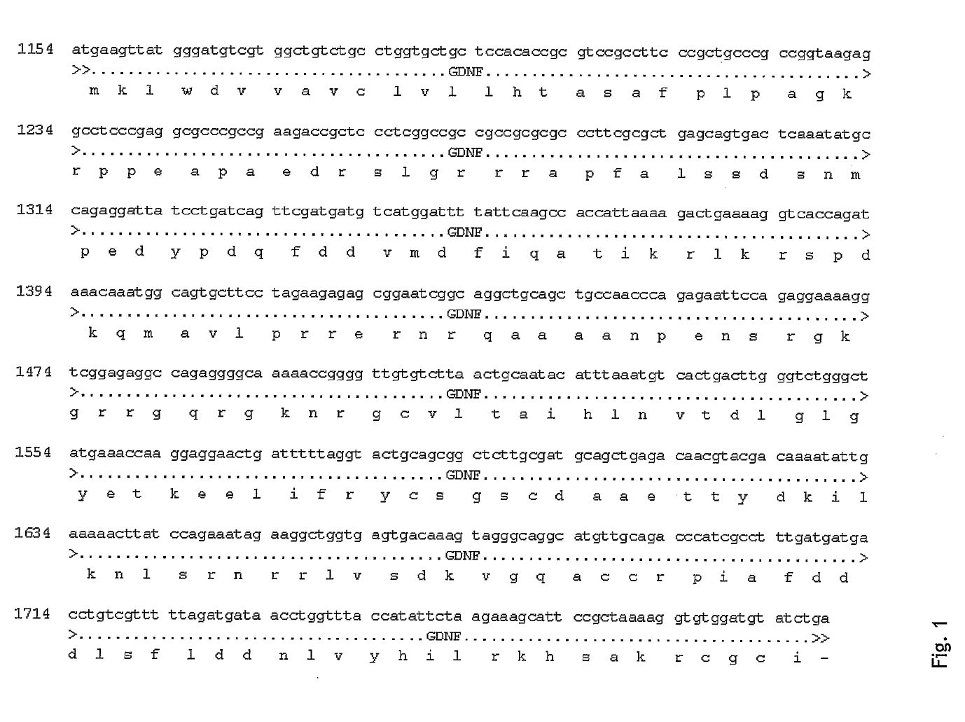

cDNA was synthesized from Caudate nucleus polyA mRNA (Clontech, Becton Dickinson, catalog number 636132) as described previously (Current Protocols in Molecular Biology). A fragment encoding long isoform of pre-pro- was amplified from cDNA by PCR using the primers rGDNFs (5′-GGTCTACGGAGACCGGATCCGAGGTGC-3′; SEQ ID No 15) and rGDNFas (5′-TCTCTGGAGCCAGGGTCAGATACATC-3′; SEQ ID No 16) essentially as described in Schaar et al. 1994 (Exp. Neurol. 130, 387-393). The resulting PCR product was purified and cloned into the SrfI site of pCRScript vector (Stratagene). Subsequently, the GDNF cDNA fragment was subcloned into the BamHI/XhoI sites in pNS1n and pCln.hNGF to yield pNS1n.hGDNF and pCln.hG, respectively. pNS1n is a customised vector derived from pcDNA3 (Invitrogen) with expression under control of the cytomegalovirus promoter carrying the neo resistance marker instead of zeo (Jensen et al 2002, J Biol Chem, 277: 41438-41447). pCln.hNGF has been described earlier in WO 2005/068498. The insert of the cloned GDNF is shown in FIG. 1.

The nucleotide sequence spanning from the first base of the CMV promoter/enhancer to the end of the primary transcript (including the GDNF coding sequence) for pCln.hG is set forth in SEQ ID NO 1. The corresponding sequence from the first base of the CMV promoter to the last base of the primary transcript (including the GDNF coding sequence) for pNS1n.hGDNF is set forth in SEQ ID NO 17.

### Growth and Transfection of Cell Lines

ARPE-19 cells (ATCC accession number CRL-2302, Dunn K C et al. ARPE-19, A human retinal pigment epithelial cell line with differentiated properties. Exp. Eye Res. 62: 155-169, 1996.) were grown in DMEM/F12 medium (REF Invitrogen) supplemented with 10% FBS (REF Hyclone) in 5% CO₂ at 37° C.

Analysis of Stable Clones Transfected with pCln.hG and pNS1n.hGDNF

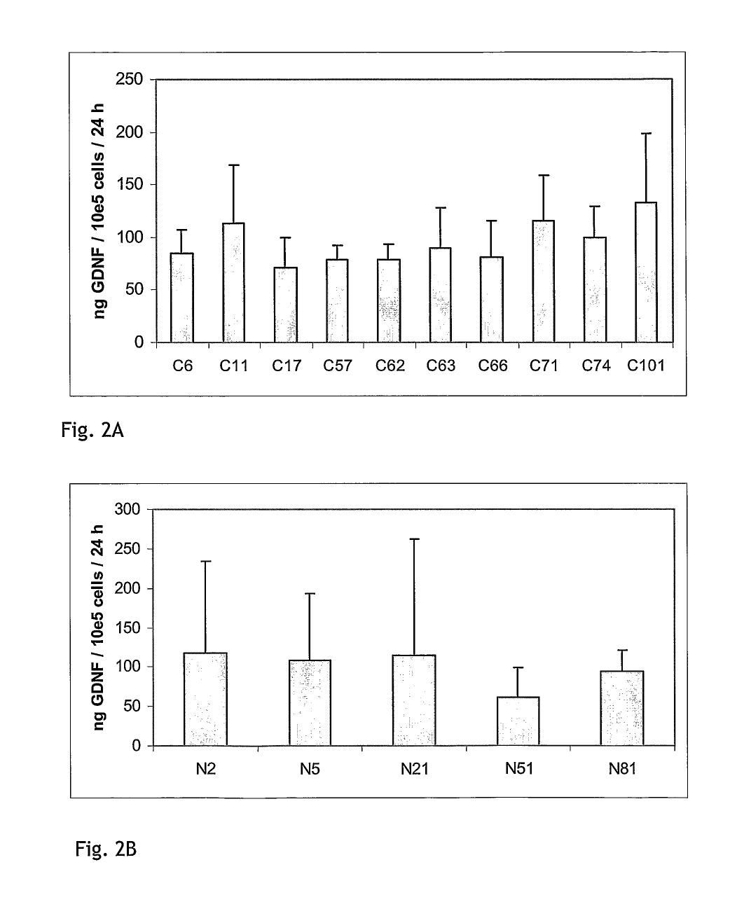

To generate recombinant cell clones, cells were transfected with linearised plasmids using FuGENE 6 Transfection Reagent (REF Roche) according to the manufacturer's recommendations. Following transfection, recombinant clones were selected in growth media supplemented with G418 (REF Sigma) and isolated using conventional cell culture techniques. A fixed number of cells were seeded in growth medium followed by media replacement after cell attachment. After 4 h incubation, media was removed and subjected to GDNF ELISA analysis using the DuoSet human GDNF ELISA kit (REF R&D systems) according to the manufacturer's recommendations. ELISA values were calculated as ng GDNF/10⁵ cells/24 h. The GDNF levels for representative clones with and without intron transfected with pCln.hG and pNS1n.hGDNF, respectively, are shown in FIGS. 2A (with intron) and 2B (without intron). pCln.hG and pNS1n.hGDNF clones were designated with first letters ‘C’ and ‘N’, respectively. All the selected clones secreted in excess of 50 ng GDNF/10⁵ cells/24 hours

Clones C101 (NGC-0301) and C63 (NGC-0363) have been deposited under the Budapest Treaty with DSMZ, Mascheroder Weg 1b, D-38124 Braunschweig, Germany, under accession numbers DSM ACC2732 and DSM ACC2733, respectively.

### Example 2

### Evaluation of GDNF Release from Confluent Stable Clones

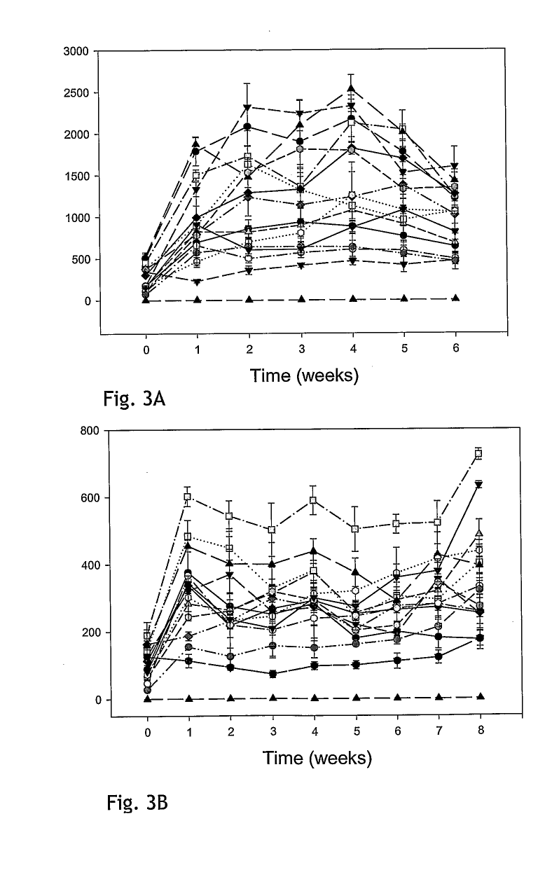

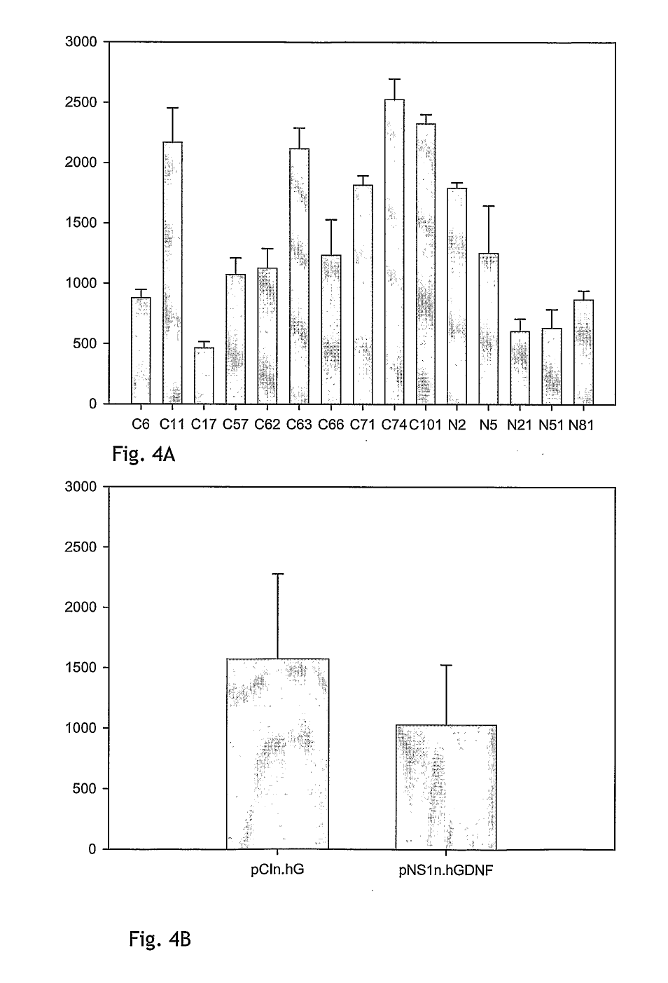

To evaluate the stability of GDNF release in confluent cultures of stable clones, a fixed number of cells were seeded in growth medium. The next day, medium was replaced with Human Endothelial Serum-free Medium (HE-SFM) from Invitrogen (cat#11111-044) or growth medium. After 4 h incubation, media were removed and subjected to GDNF ELISA analysis using the DuoSet human GDNF ELISA kit (REF RED systems) according to the manufacturer's recommendations. ELISA values were calculated as ng GDNF/ml/24 h. Cells were allowed to grow to confluency in HE-SFM or growth medium and were maintained in culture for up to 8 weeks. GDNF release in 4 h media was determined every week. The GDNF levels for representative clones transfected with the constructs pCln.hG and pNS1n.hGDNF are shown in FIGS. 3A and 3B. At the end of the experiment, cells were trypsinated and counted. FIG. 4A shows GDNF release for week 4 in HE-SFM calculated as ng GDNF/10⁵ cells/24 h. FIG. 4B shows the average of GDNF release from the two groups of constructs.

### Example 3

### Processing and Glycosylation of GDNF Secreted from ARPE-19 Cells

The purpose of this experiment is to analyse GDNF secreted from transfected ARPE-19 clones in Western blot analysis to confirm glycosylation and correct processing of the secreted GDNF.

Briefly, conditioned media from GDNF-producing ARPE-19 cells were diluted in deglycosylation reaction buffer (Prozyme Enzymatic Deglycosylation kit #GK80110) to a concentration of 0.2 ng/μl according to ELISA results. Recombinant mammalian produced hGDNF (R&D systems #212GD) was diluted to the same concentration and used as reference. Deglycosylations with and without denaturing step were performed according to protocol 3.2 and 3.3 provided by manufacturer. Samples were etectrophoresed on 8-18% gradient SDS gets. E. coli produced hGDNF (Alomone Labs #G-240) was used as a reference for non-glycosylated GDNF. Proteins were transferred to PVDF membrane. The blocked membrane was incubated with anti-GDNF antibody (R&D Systems, No AF-2,2-NA 1:500) followed by HRP-linked anti-goat antibody (1:2000) and detection with ECL (Amersham).

### Result:

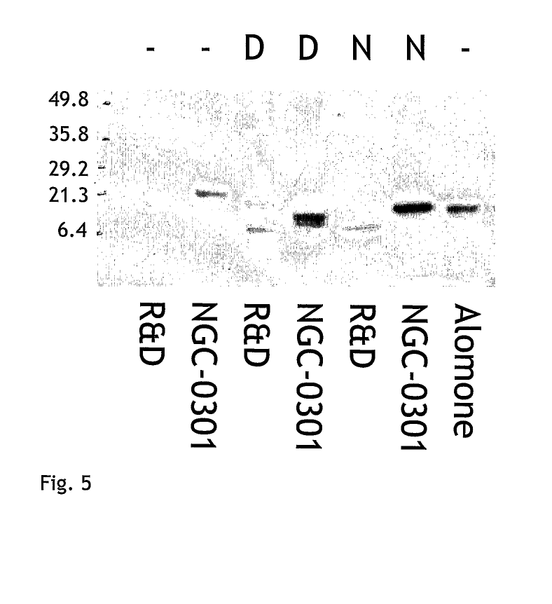

In samples with conditioned medium from GDNF producing ARPE-19 cells (NGC-0301) a band corresponding to the GDNF monomer was detected. GDNF from R&D Systems lacks 31 amino acids in N-terminal and showed an accordingly lower molecular weight (FIG. 5). Deglycosylation of ARPE-19 produced GDNF resulted in GDNF with a molecular weight of 15 kDa (same as E. coli produced GDNF) and a MW of approximately 12 kDa for R&D GDNF. The molecular weight of the band corresponding to GDNF monomer showed that the GDNF secreted from ARPE-19 cells was glycosylated and correctly processed.

### Example 4

### Bioactivity of ARPE-19 Cell-Produced GDNF Measured in Ternary Complex Formation Assay

GDNF mediates intracellular signaling and cellular responses by forming a receptor complex with the tyrosine kinase receptor Ret and the GDNF specific co-receptor GFRα1. The ability of ARPE-19 produced GDNF to form a signaling receptor complex was measured as previously described (Sanicola et al. 1997, Proc Natl Acad Sci USA 94: 6238-43). In short, GDNF was captured in plates coated with GFRα1-Ig. Subsequently, a fusion protein of Ret and Alkaline Phosphatase (Ret-AP) was added. After washing, a substrate for AP was added and color development was measured. Human bacterially produced GDNF and human mammalian produced GDNF were purchased from Alomone Labs (#G-240) and R&D Systems (#212GD), respectively, and used as controls.

### Results:

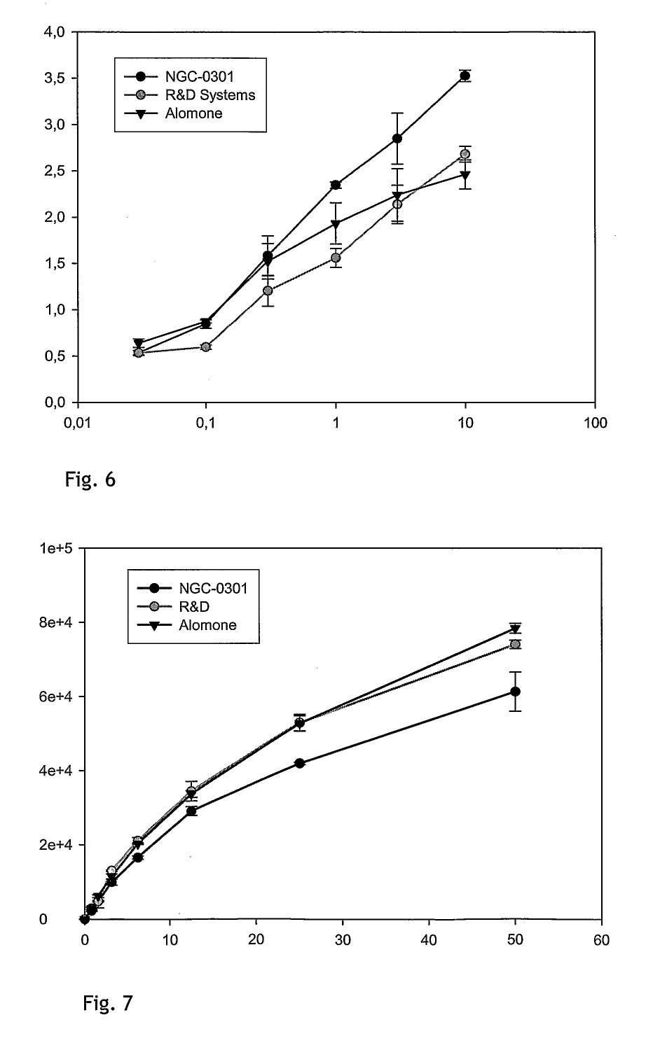

Formation of receptor complexes from ARPE-19 cells-produced GDNF was dose-dependent and comparable to GDNF from other sourcers (FIG. 6).

### Example 5

### Bioactivity of ARPE-19 Cell-Produced GDNF Measured in PC12 Cells

PC12 cells are pheochromocytoma cells expressing TrkA and have been widely used as a model system for NGF-induced differentiation. In addition, PC12 cells express the tyrosine kinase receptor Ret. In the presence of the GDNF specific co-receptor GFRα1, the formation of a GDNF-GFRα1-Ret signaling complex leads to similar cellular responses as NGF-TrkA signaling: growth arrest, extension of long neurites and a phenotype similar to sympathetic neurons. The endogenous level of GFRα1 is insufficient for a significant response, but higher GFRα1 levels can be obtained by transfection with plasmids containing cDNA for GFRα1 or by addition of GFRα1-Ig fusion protein. In the present experiment, a PC12 subclone was used for testing bioactivity of human GDNF produced by transfected ARPE-19 clones in the presence of GFRα1-Ig (R&D Systems #714-GR). Recombinant mammalian produced hGDNF (R&D systems #212GD) and E. coli produced hGDNF (Alomone Labs #G-240) were used as positive controls.

Parental ARPE-19 cells and GDNF-producing clones were seeded in T75 flasks (3×10⁶ cells per flask). The next day, cells were changed to pre-heated serum-free DMEM. After 24 h the conditioned medium was collected and centrifuged (3000×G for 5 minutes) to remove cellular debris. GDNF concentration in conditioned medium was determined by GDNF DuoSet ELISA (R&D systems #DY212) according to the manufacturer's instructions. Concentrations of R&D Systems GDNF and Alomone GDNF were adjusted according to ELISA results.

PC12 cells were cultured in Dulbecco's modified Eagle's medium (DMEM) with 4.5 g/l glucose and glutamax (Life Technologies #32430-027) with 7.5% donor horse serum (Life Technologies #16050-098) and 7.5% FBS (Life Technologies # 10099-141) in the presence of 5% CO₂ at 37° C. Medium was changed every 2-3 days and cells were subcultured 1:3-1:6 twice a week by tapping the flask and dispensing into new flasks.

PC12 cells were seeded for survival assay in 48-well dishes coated with collagen at a density of 25,000 cells/cm2 (2×10⁴ cells/well) in growth medium with serum. The next day, conditioned medium from parental ARPE-19 cells was diluted 1:2 in serum-free DMEM. This medium was used to make dilutions of GDNF from conditioned medium as well as GDNF from R&D Systems and Alomone Labs. Dilutions of GDNF were added to the PC12 cells together with 1 μg/ml Add GFRα1-Ig. Cells were incubated for three days and cell survival was determined by performing MTS assay (Promega #G5430) according to the manufacturer's instructions.

### Result:

GDNF produced from ARPE-19 cells increased MTS reduction in a dose-dependent manner. Bioactivity of ARPE-19-produced GDNF was at least as high as GDNF from other sourcers (FIG. 7).

### Example 6

### Preparation of Devices for CNS Use

Devices are fabricated from polysulphone (PS), or polyether sulfone (PES) or an equivalent polymer hollow fiber membrane with an outside diameter of 800-1000 μm and a wall thickness of approximately 100 μm. A scaffolding material consisting of polyvinyl alcohol (PVA) sponge, polyethylene (PET) yarn or similar material inserted into the membrane fiber cavity ensures proper cell distribution and attachment of the cells. Finally, a tether fabricated from polyurethane (PU) or an equivalent material fixed to the device end provides a means for device retrieval post-implantation.

Devices used for pre-clinical testing (in rats) are approximately 5-7 mm long. Devices contemplated for implantation into human brains are approximately 5-20 mm long.

Cellular loading occurs through a hub segment and port attached to the hollow fiber device at the end distal to the tether. GDNF cells prepared as a single-cell suspension are infused into the port, the hub segment is retrieved and the infusion hole is sealed with glue. For each mm length of the devices, approximately 10,000 GDNF-expressing cells are loaded. The devices are maintained in media until use.

Devices for implantation into rat brains were made with the following materials: Membrane: PS device: Polysulphone hollow fiber membrane (PS90/700 from Minntech Corp, Minneapolis, Minn., USA), with a 90 kDA molecular weight cutoff. Dimensions: 700 μm+/−50 μm inner diameter, 100 μm+/−20 μm wall. PES device: Polyethersulfone: PES5 from Akzo Nobel with a 280 kDa molecular weight cutoff. Dimensions: 520 μm+/−50 μm inner diameter, 100 μm+/−20 μm wall. Hollow fibers were cut to lengths of approximately 5 mm (PS device) and 7 mm (PES device).

Foam: PS device: PVA foam, product no. 160 LD from Hydrofera Inc, Cleveland, Ohio, USA.

PES device: Clinicel sponge from M-PACT, Eudora, Kans., USA. The PVA foam was cut to fit the inner diameter of the hollow fiber.

Load tube: Perfluoroalkoxy copolymer. Dimensions: PS device: 0.0037″+/−0.0005″ ID; 0.005″+/−0.001″ wall. PES device: 410 μm+/−50 μm ID; 45 μm+/−5 μm wall. Both from Zeus Industrial Products, Orangeburg, S.C., USA. The load tube is glued to the hollow fiber in one end and to the hub in the other end.

Hub: Product no P/N 02030200 Rev 1, from Abtec, Bristol, Pa., USA.

Glue for gluing load tube to hub: Dymax 201-CTH (Diatom, Hvidovre, Denmark).

Glue for hollow fiber: PS device: Dymax 1181-M. PES device: Dymax 1188-M.

Devices were assembled in a controlled environment, packaged in Falcon 15 mL polypropylene test tubes (Becton Dickinson, Cat #352096) and sterilised by exposure to ethylene oxide prior to filling with cells.

### Example 7

### In Vitro Testing of Encapsulated GDNF-Producing ARPE-19 Cell Clones

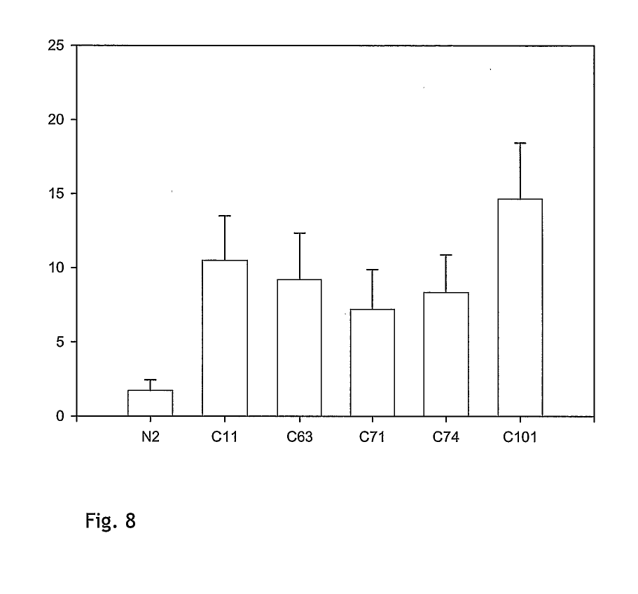

Cells were expanded in growth media as described above. The day before encapsulation, the cells were trypsinized, counted and re-seeded in growth media. Immediately before encapsulation, cells were trypsinized, counted, washed and resuspended in Human Endothelial Serum Free Media (HE-SFM, Invitrogen). The suspension was kept at room temperature through the experiment. Devices were made according to the specifications in Example 6. Cells were carefully injected into the device using a Hamilton syringe. For the experimental devices, 50,000 cells in 8 μl were injected in each device. After cell encapsulation and device sealing, the devices were transferred to HE-SFM. Remaining cells were seeded in growth media in conventional cell culture dishes to confirm cell viability. Throughout the experiment, the devices were maintained in HE-SFM at 37° C./5% CO₂. Samples were taken at desired time points for GDNF ELISA: Devices were washed in HE-SFM and subsequently incubated in 1 ml HE-SFM for 4 h. Released GDNF was measured using the DuoSet Human GDNF-ELISA (R&D systems). The amount of secreted GDNF per device is shown in FIG. 8.

After 14 days in vitro, devices were fixed in paraformaldehyde, embedded and sectioned, and eosin/hematoxylin-stained using standard histology techniques. Cell survival was determined from device sections. The device sections confirmed that in vitro survival was excellent.

### Example 8

### In Vivo Testing of Encapsulated GDNF-Producing ARPE-19 Cell Clones

14 days before implantation, cells were encapsulated as described in Example 6. Samples of clone C11, C63, C71, C74, C100 and clone N2 were encapsulated. Until implantation, the encapsulated cells were maintained in HE-SFM at 37° C./5% CO₂. One lot of devices was maintained in vitro for ten weeks. GDNF-release (for 1 hour) was measured twice a week as described elsewhere. Medium was exchanged in connection with the GDNF release measurements. After two weeks in vitro, one lot of devices were implanted in rat brains.

Adult, 220 gram female Sprague-Dawley rats housed under 12 hours light:dark cycle and with free access to rat chow and water were used for implantation surgery. Devices were implanted bilaterally in striatum in isofluoran anesthesised (1.5-2%) animals in the site described by the following coordinates with respect to bregma: AP: 0.0, ML: +/−3.2, DV: −8, TB: 3.3. Following surgery, assessment of general behaviour and weight was monitored every other week. At 8 weeks post implantation animals were decapitated. Devices were removed from the brain and rinsed once in PBS. The PBS was replaced with 1 ml Serum-Free Medium and incubated for GDNF release measurement as described previously. Devices were subsequently fixed in 4% paraformaldehyde and processed for histological analysis by hematoxylin & eosin staining. The brain was dissected out and rinsed in cold saline for 1 minute before preparation in 4% paraformaldehyde and immersion fixation overnight at 4° C. Brains were then transferred into 25% sucrose/0.1M phosphate buffer and after 48 hours, 40 μm sections were cut on a freezing microtome. Brain sections were subsequently processed for general morphology (hematoxylin a eosin, cresyl violet).

Weight and behaviour of the rats was normal and did not differ among the different treatments.

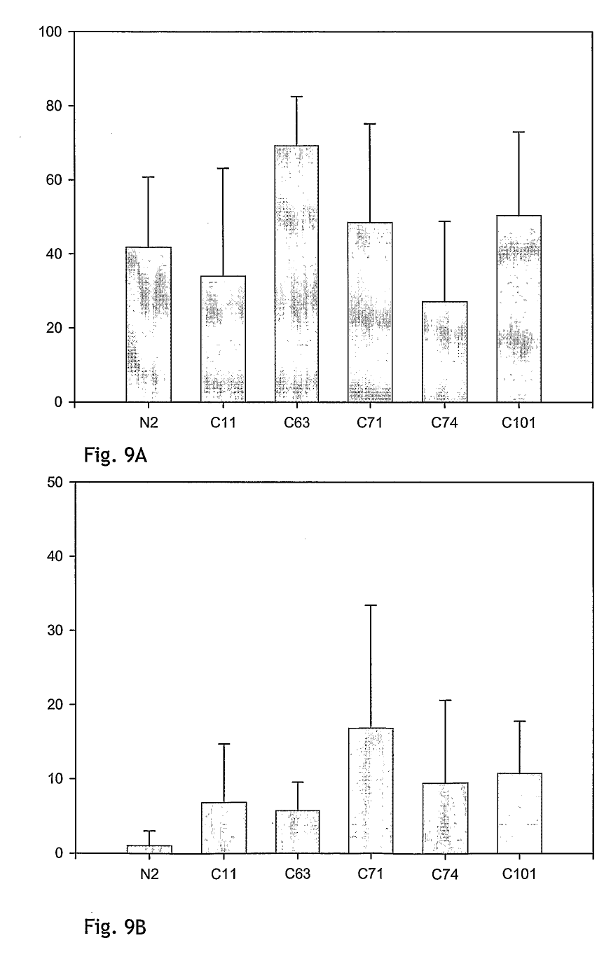

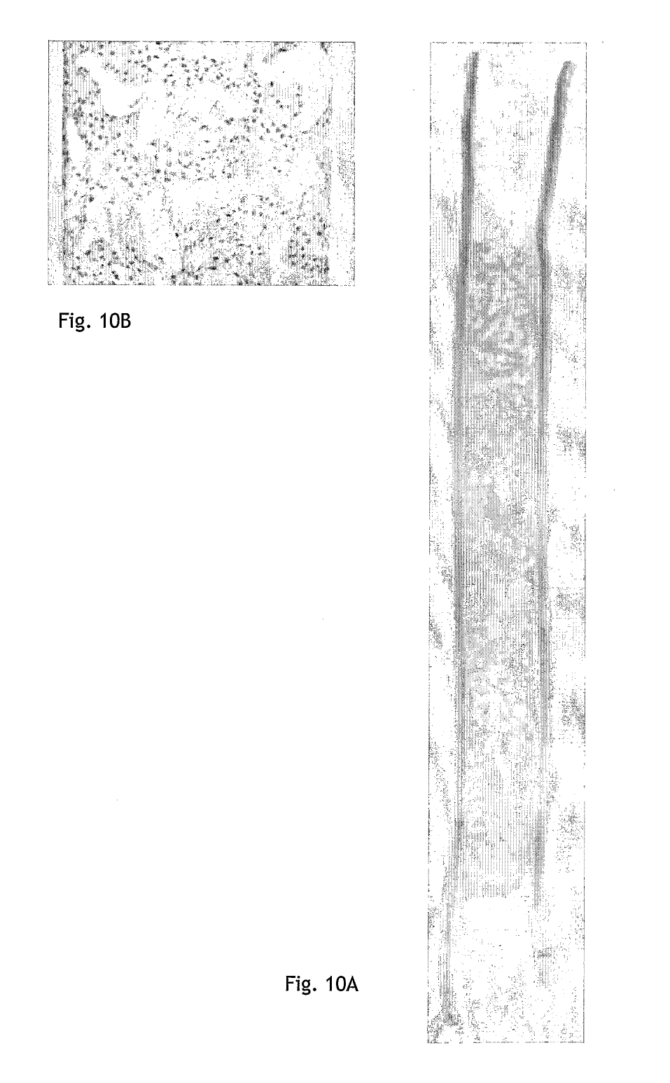

Results depicted in FIG. 10 show prominent cell survival after 8 weeks in normal rats in both PS and PES devices. GDNF secretion from explanted devices was as high as pre-implantation secretion, and appeared to be higher from the PES devices (FIG. 9A) than from the PS devices (FIG. 9B).

### Example 9

### Intrathecal Implantation of Devices

Intrathecal implantation can be accomplished along the spinal canal, preferably at the lumbar level below the conus medullaris in e.g. human beings. A small incision is made at the lumbar level, and a spinal needle is used to enter the intrathecal space. After CSF flow has been established, a guide wire is inserted into the intrathecal space and a dilator system is used to enter the space. The guidewire is withdrawn and the encapsulated device inserted into the space so that the active part is completely enclosed in the CSF compartment. The tether is secured to the lumbar fascia by a non-resorbable suture and preferably using a securing clip. The skin is closed using standard surgical procedures.

### Example 10

### Implantation in the Human Striatal Structures

Under general anesthesia or local anesthesia and sedation, a neurosurgical stereotactic frame is secured to the patient's head. A fiducial box and subsequent MRI imaging is applied to determine the anatomical area and implantation coordinates. The implantation can also be guided by diffusion tensor imaging and dose mapping, utilising custom software and navigational equipment supplied by BrainLAB AG. The patient is next brought to the operating room where he/she is prepped and draped. Based on the stereotactic image data a small skin incision is made frontolaterally and a small burrhole made through the skull. The dura and underlying meninges are penetrated by incision and a guide cannula with a trochar is inserted into the putamen and caudate nucleus target area. The trocar is removed and the device is slided into position. The guide is removed and the device tether secured to the skull with a titanium plate or custom retaining clip. One or more devices may be inserted into the same structure. The skin is sutured closed with interrupted 3-0 Nylon suture. The procedure is repeated on the opposite side.

1. A biocompatible device comprising an inner core comprising a composition of human cells comprising a heterologous expression construct coding for GDNF, and a semipermeable membrane surrounding the composition of cells, said membrane permitting the diffusion of GDNF, wherein the human cells are from a monoclonal cell line.

2. (canceled)

3. The device of claim 1, wherein the cells are transfected with a non-viral expression construct.

4. The device of claim 1, wherein the expression construct is a plasmid.

5. The device of claim 1, wherein the human cells are contact inhibited cells.

6. The device of claim 1, wherein the cells are capable of phagocytising.

7. The device of claim 1, wherein the cells are non-tumourigenic.

8. The device of claim 1, wherein the cells have been immortalised by insertion of a heterologous immortalisation gene.

9. The device of claim 1, wherein the cell line has not been immortalised by insertion of a heterologous immortalisation gene.

10. The device of claim 1, wherein the cell line is non-tumourigenic in mammals, preferably in human beings.

11. The device of claim 1, wherein the cell line is able to survive at low oxygen tension, such as at less than 5% oxygen tension, more preferably less than 2%, more preferably less than 1%.

12. The device of claim 1, wherein the cell line originates from a primary culture.

13. The device of claim 1, wherein the cell line is capable of undergoing at least 50 doublings, more preferably at least 60, more preferably at least 70, more preferably at least 80, more preferably at least 90, more preferably at least 100.

14. The device of claim 1, wherein the cell line triggers a low level of human host immune reaction, preferably wherein the human antibody and/or complement dependent cytotoxicity is lower in humans than in a non-human animal.

15. The device of claim 1, wherein the cells are derived from an epithelial cell.

16. The device of claim 15, wherein the epithelial cell is a retinal pigment epithelial cell.

17. The device of claim 1, wherein the expression construct comprises an intron in the transcript.

18. The device of claim 17, wherein the intron is located in the 5′ UTR of the transcript.

19. The device of claim 1, wherein the device is capable of secreting in excess of 1 ng GDNF/24 hours.

20. The device of claim 1, comprising between 10,000 and 100,000 cells per μL of device, more preferably from 15,000 to 50,000 cells per μL, more preferably from 20,000 to 30,000 cells per μL.

21. The device of claim 1, having a volume of at least 0.5 μL, preferably at least 1 μL.

22. The device of claim 1, containing substantially less than 10⁴ cells, such as less than 1000 cells per capsule for example less than 100 cells per capsule, such as less than 50 cells per capsule, for example less than 10 cells per capsule, such as less than 5 cells per capsule.

23. The device of claim 1, having a diameter of less than 250 μm, such as less than 150 μm, for example less than 100 μm, such as less than 50 μm, for example less than 25 μm.

24. The device of claim 1, wherein the core comprises a support for the cells.

25. The device of claim 1, wherein the semipermeable membrane is capable of preventing cell-cell contact between cells in the core and cells outside the device.

26. The device of claim 25, wherein the membrane has a molecular weight cutoff of 300 kDa or less.

27. The device of claim 1, further comprising a tether.

28. The device of claim 1, further comprising a suture eyelet.

29. The device of claim 1, comprising cells obtainable from the cell line deposited with DSMZ under accession number DSM ACC2732 or DSM ACC2733.

30. A composition of human cells derived from a monoclonal cell line, wherein the cells comprise a heterologous expression construct coding for GDNF.

31. The composition of claim 30, wherein the secreted GDNF has the amino acid sequence of SEQ ID NO 9.

32. The composition of claim 30, comprising cells obtainable from the cell line deposited with DSMZ under accession number DSM ACC2732 or DSM ACC2733.

33. The composition of claim 30, wherein the cells are cultured on a support-matrix.

34. A method of treatment of Parkinson's disease comprising implanting the device of claim 1 into the putamen and striatal structures of a subject in need thereof.

35. A method of treatment of Amytrophic Lateral Sclerosis comprising implanting the device of claim 1 into the intrathecal space and/or the spinal cord of a subject in need thereof.

36. A method of treatment of Huntington's Disease comprising implanting the device of claim 1 into the putamen and striatal structures of a subject in need thereof.

37. A method of treatment of retinopathy, age-related macular degeneration, glaucoma, ocular neovascularisation or retinal degeneration comprising implanting the device of claim 1 into the eye of a subject in need thereof.

38. A method of treatment of Parkinson's disease comprising implanting the composition of cells according to claim 30 into the putamen and striatal structures of a subject in need thereof.

39. A method of treatment of Amytrophic Lateral Sclerosis comprising implanting the composition of cells according to claim 30 into the intrathecal space and/or the spinal cord of a subject in need thereof.

40. A method of treatment of Huntington's Disease comprising implanting the composition of cells according to claim 30 into the putamen and striatal structures of a subject in need thereof.

41. A method of treatment of retinopathy, age-related macular degeneration, glaucoma, ocular neovascularisation or retinal degeneration comprising implanting the composition of cells according to claim 30 into the eye of a subject in need thereof.
